(19) United States
(12) Patent Application Publication
(10) Pub. No.: US 2024/0065576 A1
(43) Pub. Date: Feb. 29, 2024

(54) PHYSIOLOGICAL PARAMETER RELIABILITY INDICATORS

(71) Applicant: Stryker Corporation, Kalamazoo, MI (US)

(72) Inventors: Fred W. Chapman, Newcastle, WA (US); Tyson G. Taylor, Bothell, WA (US); Doan Huu Dinh, Seattle, WA (US)

(73) Assignee: Stryker Corporation, Kalamazoo, MI (US)

(21) Appl. No.: 18/239,377

(22) Filed: Aug. 29, 2023

Related U.S. Application Data
(60) Provisional application No. 63/401,983, filed on Aug. 29, 2022.

(51) Int. Cl. A61B 5/083; A61B 5/08

(52) U.S. Cl. CPC A61B 5/0836; A61B 5/0816; A61B 5/7425

(57) ABSTRACT

Systems and methods described herein relate to determining physiological parameters and associated reliability of the physiological parameters and displaying the data in a manner that conveys the reliability information to a user. The reliability index is determined and then used to determine one or more display attributes for displaying the underlying parameter data while also conveying the reliability information. The implementations described herein provide for displaying physiological parameters in a manner that is easily digested and understood while also conveying a reliability in such data, without filling or cluttering a display with background or additional data that is typically used in a manual method to evaluate the data on the display.

### CROSS-REFERENCE TO RELATED APPLICATION

This application claims priority to U.S. Provisional App. No. 63/401,983, filed on Aug. 29, 2022, which is incorporated by reference herein in its entirety.

### BACKGROUND

Medical device monitors have limited screen estate that is used to showcase multiple vital sign parameters. Showing additional data and additional information can consume this valuable screen estate and may reduce a user's access to patient information.

Physiological parameter monitoring devices display data regardless of whether the data is accurate or not. Currently, users visually assess whether data is accurate or is skewed as a result of artifacts or other noise.

However, artifacts and noise can lead to erroneous measurements that users do not appreciate using visual assessments. In For example, a vital sign monitor determines a heart rate of a patient by detecting QRS complexes in electrocardiogram (ECG) data. But if the ECG includes significant artifact, the QRS detection is inconsistent, which leads to an inaccurate heart rate measurement. In instances where the user is inexperienced or is distracted and unable to recognize that the artifact in the ECG, the patient's care could be compromised if the user relies on the inaccurate heart rate measurement. Similar problems exist with other measured and derived physiological parameters, such as measurements of respiratory rate, NIBP, oxygen saturation, among others.

Typical monitors and devices display an indication of “no data” when a physiological parameter (e.g., respiration rate) drops or stops. However, users is unable to determine whether the indication means that the monitor has malfunctioned or if the physiological parameter has ceased.

### BRIEF DESCRIPTION OF THE DRAWINGS

FIG. 1 illustrates an example system for determining and displaying reliability indicators for physiological parameters, according to at least one example.

FIG. 2 is an example block diagram of a physiological feedback system with reliability indicators, according to at least one example.

FIG. 3 is an example block diagram of a system for monitoring physiological parameters and providing indication of reliability, according to at least one example.

FIG. 4 is an example block diagram of a physiological monitoring system with a display for outputting the parameter and reliability of the parameter, according to at least one example.

FIG. 5 illustrates an example chart showing information associated with automated or manual rhythm interpretation while chest compressions are ongoing, according to at least one example.

FIG. 6 illustrates a representative scale indicating association between reliability of a physiological parameter and variability in the reliability indicator, according to at least one example.

FIG. 7A illustrates an example display of oxygen saturation having a first reliability of the physiological data, according to at least one example.

FIG. 7B illustrates an example display of oxygen saturation having a second reliability of the physiological data, according to at least one example.

FIG. 8 illustrates an example of an external defibrillator configured to perform various functions described herein, according to at least one example.

FIG. 9 illustrates an example of a capnography device configured to perform various functions described herein, according to at least one example.

FIG. 10 is an example flowchart illustrating a method for determining to display a first physiological parameter or second physiological parameter on a display associated with a medical device, according to at least one example.

FIG. 11 is an example flowchart illustrating a method for displaying a reliability indication of physiological parameters, according to at least one example.

### DETAILED DESCRIPTION

Various implementations described herein relate to determining physiological parameters and associated reliability of the physiological parameters and displaying the data in a manner that conveys the information to a user with minimal distraction. The physiological parameters monitored by one or more devices, such as those described herein provide data with detail and information that is burdensome to a user, or lead to an incorrect conclusion if the data is not properly assessed and presented. Accordingly, presenting physiological parameter data to a user in a manner that provides information related to the parameter itself as well as the reliability or accuracy of the measurement aids a user in making decisions for treatment in real-time.

The implementations described herein provide for displaying physiological parameters in a manner that is easily digested and understood while also conveying a reliability of such data, without filling or cluttering a display with background or additional data that is typically used in a manual method to evaluate the data on the display.

Various implementations described herein are directed to specific improvements in the technical field of emergency medical devices. For instance, by displaying reliability indicators regarding the accuracy and reliability of the displayed parameters, thereby providing technical improvements to how emergency medical devices evaluate and provide information in emergency situations.

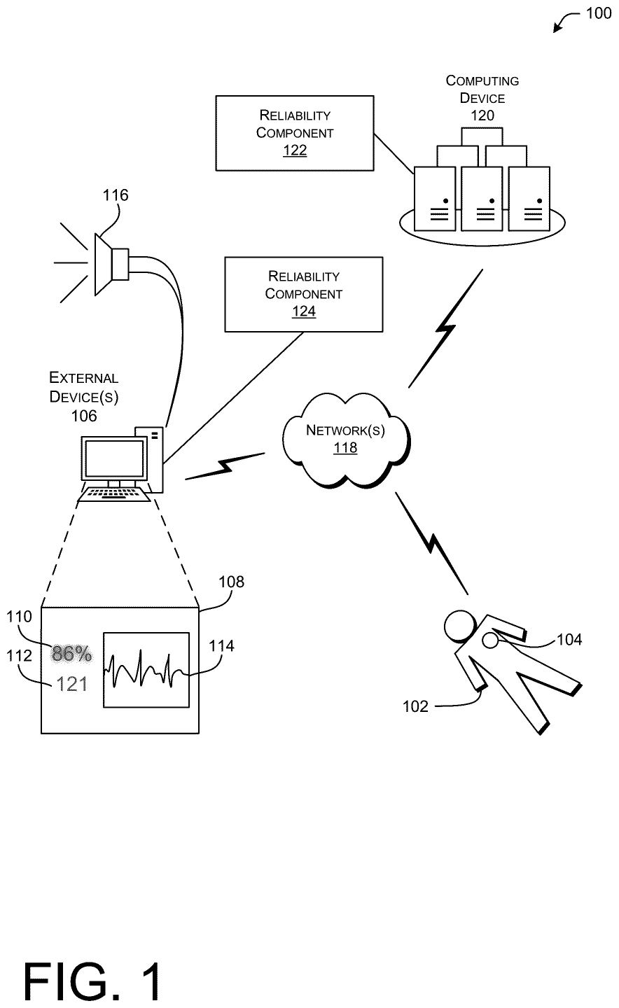

FIG. 1 illustrates a system 100 for determining and displaying reliability indicators for physiological parameters, according to at least one example. In some implementations, the system 100 includes a sensor 104 configured to detect a physiological parameter of a patient 102. In some implementations, the sensor 104 includes a variety of different sensors, including respiration sensors, cardiac sensors, oxygenation sensors, capnography sensors, and other such devices configured to detect one or more physiological parameters of the patient 102. In some examples the physiological parameter includes ECG data or other non-ECG data (e.g., a heart rate level or waveform, a temperature level or waveform, an airway CO₂ level or waveform, an oxygenation level or waveform, a blood pressure level or waveform, etc.). In some examples, the sensor 104 is configured to communicate over a network 118, which includes wired and/or wireless connections, to an external device 106, such as a computing device having a processor and processor executable instructions to perform various actions, where information is displayed on a display 108 for observation and analysis by a user. The sensor 104 and/or external device 106 is further in communication over the network 118 (which, in some examples, includes one or more additional networks) with a computing device 120 that is remote from the sensor 104 and/or the external device 106. The computing device 120 performs one or more calculations and/or determinations based on data from the sensor 104, for example to display at the display 108.

In some examples, the display 108 includes representations of physiological parameters that include numerical representations, charts, graphs, or other such representations. In an example, the display 108 includes a representation of oxygenation 110, pulse 121, heart rate, and a representation of an electrocardiogram (ECG) signal 114.

As data is received from the sensor 104, the external device 106, through the reliability component 124, or through a reliability component 122 of the computing device 120, determines a reliability of the data. In some examples, the reliability is determined based on noise within the data or other such parameters of the data. In some examples, a physiological parameter is determined from one or more signals detected by the sensor 104 and/or other sensors. In such examples the reliability component 122 and/or reliability component 124 determines a reliability of the physiological parameter based on the underlying signals from the sensor 104. In some examples, as discussed herein, the reliability of the physiological parameter is based on inputting the signals from the sensor 104 into a machine learning model (e.g., a neural network) trained to output a reliability score associated with the physiological parameter determined using those signals. As can be understood in the context of this disclosure, a neural network can utilize machine-learning, which can refer to a broad class of such algorithms in which an output is generated based on learned parameters.

Although discussed in the context of neural networks, any type of machine-learning can be used consistent with this disclosure. For example, machine-learning algorithms can include, but are not limited to, regression algorithms (e.g., ordinary least squares regression (OLSR), linear regression, logistic regression, stepwise regression, multivariate adaptive regression splines (MARS), locally estimated scatterplot smoothing (LOESS)), instance-based algorithms (e.g., ridge regression, least absolute shrinkage and selection operator (LASSO), elastic net, least-angle regression (LARS)), decisions tree algorithms (e.g., classification and regression tree (CART), iterative dichotomiser 3 (ID3), Chi-squared automatic interaction detection (CHAID), decision stump, conditional decision trees), Bayesian algorithms (e.g., naïve Bayes, Gaussian naïve Bayes, multinomial naïve Bayes, average one-dependence estimators (AODE), Bayesian belief network (BNN), Bayesian networks), clustering algorithms (e.g., k-means, k-medians, expectation maximization (EM), hierarchical clustering), association rule learning algorithms (e.g., perceptron, back-propagation, hopfield network, Radial Basis Function Network (RBFN)), deep learning algorithms (e.g., Deep Boltzmann Machine (DBM), Deep Belief Networks (DBN), Convolutional Neural Network (CNN), Stacked Auto-Encoders), Dimensionality Reduction Algorithms (e.g., Principal Component Analysis (PCA), Principal Component Regression (PCR), Partial Least Squares Regression (PLSR), Sammon Mapping, Multidimensional Scaling (MDS), Projection Pursuit, Linear Discriminant Analysis (LDA), Mixture Discriminant Analysis (MDA), Quadratic Discriminant Analysis (QDA), Flexible Discriminant Analysis (FDA)), Ensemble Algorithms (e.g., Boosting, Bootstrapped Aggregation (Bagging), AdaBoost, Stacked Generalization (blending), Gradient Boosting Machines (GBM), Gradient Boosted Regression Trees (GBRT), Random Forest), SVM (support vector machine), supervised learning, unsupervised learning, semi-supervised learning, etc. Additional examples of architectures include neural networks such as ResNet-50, ResNet-101, VGG, DenseNet, PointNet, and the like. In some examples, the ML model discussed herein may comprise PointPillars, SECOND, top-down feature layers (e.g., see U.S. patent application Ser. No. 15/963,833, which is incorporated in its entirety herein), and/or VoxelNet. Architecture latency optimizations may include MobilenetV2, Shufflenet, Channelnet, Peleenet, and/or the like. The ML model may comprise a residual block such as Pixor, in some examples.

In some examples, the reliability component 122 and/or the reliability component 124 determines a reliability score for the physiological parameter(s) displayed on the display 108 using data from the sensors and analysis of the signals. For example, data from an oxygen saturation sensor is analyzed to determine a reliability of a blood oxygen saturation parameter. The analysis of the data includes waveform morphology analysis of the sensor data from a first beat (e.g., heartbeat) to a second beat, and/or over one or more periods of time. In some examples, heart pulse events is determined from the oxygenation waveform and is correlated with heart pulse events (e.g., heartbeats) determined from a second set of data, such as an ECG waveform. In some examples, reliability is based on a morphology analysis of capnograph waveforms.

After reliability of the parameters are determined, one or more display settings is adjusted to reflect the reliability of the data and/or the physiological parameters. In an example, the display setting includes changing a brightness, contrast, and/or a color of a portion of the display 108, such as the portion of the display showing the particular parameter. In a particular example, a brightness of a portion of the display 108 showing a blood oxygenation value is dimmed or have reduced contrast in response to determining that the reliability of the blood oxygenation value is below a threshold level. In such examples, the adjustment of the display setting causes the display of the parameter to be less visible to a user, and thereby indicate the reduced or diminished reliability of the value.

In some examples, the display setting includes a blurriness of the particular value on the display 108. As depicted in FIG. 1, a particular value, such as the oxygenation 110 has a reliability score determined by the reliability component 124 below a threshold level, and therefore is blurred. The blurriness scales proportional to the reliability score associated with the parameter. For instance, a blurring parameter describing an amount of blur to apply to the display of the value scales linearly with the inverse of the reliability score such that as the reliability score increases the blurriness decreases. The reliability score, in some examples, relates to an output from a machine learning model. The model outputs the reliability score with a lower reliability score that is the same as or may reflect a lower reliability of the value. The amount of blur, in some examples, relates to a reduction in pixels used to display the indicators. In some examples, the amount of blur may be related to a distance from a center of the indicator to the edges of the indicator, e.g., a blur distance may increase as the blur amount increases, the blur distance describing a distance from a center to an edge of a numeral or other reference of the indicator. In some examples, a sharpness is used to measure the blurriness of the value, with an increased sharpness of the display indicative of an increased reliability score.

In some examples, the display setting includes adjusting an output of a graph showing a representation of the data, such as the ECG data 114. In some examples, the data displayed on the graph is determined from one or more signals from the sensor 104 and is therefore not be a direct output of sensor data. In such examples, in response to the reliability being below a threshold level for a threshold period of time, the underlying sensor data is display in place of the determined data. In some examples, the graph is also adjusted to display additional and/or fewer signals, such as to remove representations of parameters having low reliability scores and/or to remove representations of derived parameters when underlying signals used to determine the derived parameters have reliability scores below a threshold level. In some examples, the graphs is adjusted according to one or more display settings to adjust a clarity or visibility of one or more representations of the data on the graph. For example, a line thickness, line color, pattern representing a line, blurriness, or other setting for the representation within the graph is adjusted to reflect the reliability score, with more visible representations indicative of higher levels of reliability generally.

In some examples, the display setting includes an speaker 116. The audio output scales, similar to the blurriness, according to the reliability score. For instance, a tone or white noise sound (e.g., a noise containing many frequencies with equal intensities) is played with a volume that scales according to the reliability score, with a higher volume associated with a lower reliability score in the parameter. In some examples, combinations of tones from multiple different parameters is combined into an overall reliability tone for the display 108 that reflects the overall reliability of the data on the display 108. In some examples, the display settings is focused on individual values or on the entire display 108, and is toggled by a user, for example who wishes to focus on a particular parameter.

In an example, the sensor 104 includes a capnography sensor configured to detect a capnograph of the patient 102. A second sensor, or component of sensor 104 (not separately pictured in FIG. 1) detects a respiration rate of the patient. The sensor 104 conveys data associated with the capnography sensor and the respiration rate sensor to the computing device 120 and/or the external device 106 where a physiological parameter of the patient 102 is determined using the data from the sensor 104. For instance, the external device 106 determines an end-tidal carbon dioxide parameter for the patient 102 by analyzing the capnograph data. The end-tidal carbon dioxide is displayed on the display 108. The external device 106 further causes the display 108 to show the respiration rate data. In the example, the respiration rate drops below a threshold level for a threshold period of time, which is a result of sensor error or a result of slowing or stopping respiration of the patient—very different clinical conditions requiring different actions on different timeframes that need to be distinguished to determine if intervention with the patient 102 is warranted. For example, when the respiration rate of the patient 102, as detected, drops below five breaths per minute over a threshold period of time of ten to thirty seconds or more. In some examples, other thresholds, such as higher or lower breaths per minute over greater or shorter periods of time is introduced as well. The drop in the respiration rate is associated with a drop in a reliability score for the end-tidal carbon dioxide, or other parameters, as the drop in the respiration rate is a sign of unreliable data. Accordingly, the external device 106 causes the display 108 to output a representation of the capnograph, rather than the derived end-tidal carbon dioxide value.

In some examples, the end-tidal carbon dioxide value is removed from the display 108 in response to determining the reliability of the data is below a threshold value for a threshold period of time. For example, the end-tidal carbon dioxide is removed from the display 108 and replaced with a representation of the capnograph data from the sensor 104. The capnograph data is displayed as a chart, graph, numerical value, or other representation, with an indicator that the display 108 is presenting capnograph data rather than end-tidal carbon dioxide. In some examples, the display of the end-tidal carbon dioxide and the capnograph data is presented on the display 108 in different types of representations (e.g., a numerical value versus a graphical representation) to enable a user to rapidly identify the type of data displayed. In such examples, the capnograph data is presented over a period of time, rather than at an instant point in time, with the capnograph representing data detected at a present time and a previous time while the end-tidal carbon dioxide is presented as an instantaneous numerical value associated with a present value.

In the example, the display 108 additionally has a feature altered to reflect the change in data presented on the display 108. For instance, when the capnograph data is presented on the display rather than the end-tidal carbon dioxide, a color of a portion of the display associated with the representation changes, such as to a bright or highly visible color such as red or yellow to draw attention to the change in the displayed data. In some examples, the brightness of the display is also changed, across the entire display, such as to increase a brightness when the capnograph data is presented, and/or by changing a label of data on the display to label the capnograph data as such.

In some examples, the external device 106 further causes the speaker 116 to output an audio signal. The audio signal is altered in response to presenting the capnograph on the display 108. For instance, the audio signal includes a tone played by the speaker 116 when the capnograph is initially presented. In some examples, the audio signal is a tone that changes in pitch and/or volume in response to displaying the capnograph.

In a second example, the external device 106 and the sensor 104 together constitute a medical device for detecting a first physiological parameter of the patient 102. In the second example, the first physiological parameter is used to determine a second physiological parameter, e.g., the second physiological parameter is derived, at least in part, based on the first physiological parameter. In the second example, the first physiological parameter may be ECG data and the second physiological parameter may be heart rate data. The external device 106 causes the display 108 to visually output a representation of the second physiological parameter. The external device 106 and/or the computing device 120 determines, using the reliability component 122 and/or reliability component 124, that the second physiological parameter has dropped below a threshold level for a threshold period of time, with the threshold level and threshold period of time selected based on expected values for the second physiological parameter during normal monitoring of typical patient values.

In some examples, the reliability component 122 and/or reliability component 124 determines a reliability score for the second physiological parameter, that is based on the threshold level and threshold score. In some examples, the reliability score, in some examples, is based on the first physiological parameter, such as based on noise or volatility detected within the first physiological parameter. In some examples, the reliability score is determined by a machine learning model trained to output reliability scores for the second physiological parameter based on receiving inputs of the first physiological parameter and/or the second physiological parameter, which includes inputs of the parameters over a period of time. In response to determining that the second physiological parameter is below the threshold level for the threshold period of time, the external device 106 causes the display to visually output the first physiological parameter and remove the second physiological parameter from the display 108. In some examples, the external device 106 performs the action based on the reliability score, as discussed herein. Further, the external device 106 causes one or more additional changes to the display 108 including altering display settings, causing an audio signal to be output by the speaker 116, or other such changes as discussed herein.

In some examples, the second physiological parameter is determined using a first subset of data from the first physiological parameter. For example, the sensor 104 includes a sensor array and gathers multiple types of data, with a subset of the data used to determine the second physiological parameter. In some examples, the data displayed on the display 108 in response to the second physiological parameter being below the threshold level and/or the reliability score being below a threshold level includes a second subset of the first physiological parameter, different from the subset used to determine the second physiological parameter. In this manner, if a sensor error is present affecting the first subset, then displaying the second subset avoids displaying data affected by the sensor error.

In a third example, the external device 106 communicates with the sensor 104 to detect a physiological parameter of a patient 102. The external device 106, and/or the computing device 120 determines, using the reliability component 122 and/or reliability component 124, a reliability index of the physiological parameter. The reliability index is a score associated with an accuracy and/or a predicted accuracy of the physiological parameter. The external device 106 determines a visibility setting for the physiological parameter on the display 108 and cause the parameter to be displayed using the display setting. The visibility setting reflects a visual clarity of the representation of the physiological parameter on the display 108.

In some examples, the reliability component 122 and/or the reliability component 124 determines the reliability index by cross-correlating a first subset of the physiological parameter data with a second subset of the physiological parameter. The cross-correlation is a measure of a similarity of the two subsets and in some examples is performed using a convolution, inner product, correlation matrix, or other such calculations of cross-correlation. In some examples, the reliability component 122 and/or the reliability component 124 includes a machine learning model that is trained to output the reliability index in response to input of the physiological parameter and/or sensor data from the sensor 104 used to derive the physiological parameter.

In some examples, the visibility setting for the display 108 includes a visual sharpness and/or blurriness for the representation of the physiological parameter. The visual sharpness scales proportionally with the reliability index such that a lower reliability index is associated with a lower visual sharpness (e.g., a higher blurriness) and a higher reliability index is associated with a higher visual sharpness (e.g., a lower blurriness). The visibility setting also includes a color of the display 108, a color of a portion of the display 108, a color of text or numerical representations within the display 108, a color of a graphical representation, a brightness level of the display 108, a brightness of a portion of the display 108, a brightness of text, a brightness of a line of a graphical representation, a contrast level for the display and/or for a text or graphical item, a text size, a pixilation level (e.g. the degree to which text and/or graphical representations appear pixelated, similar to a blurriness), or other such display setting. In some examples, the visibility setting is associated with an audio signal output by the speaker 116 as described herein. The audio signal is scaled similar to the visibility setting, for example to scale a volume, pitch, proportion of static within the audio signal, frequency, tone, or other setting of the audio signal output by the speaker 116.

The visibility setting and/or display settings used by the external device 106 to display the physiological parameter is used as an indication to a user of the reliability index without presenting an additional numerical value or representation of the reliability index. In this manner, the display 108 may not be further cluttered with additional data, but instead present additional dimensions of the already presented data in an intuitive manner for users to understand.

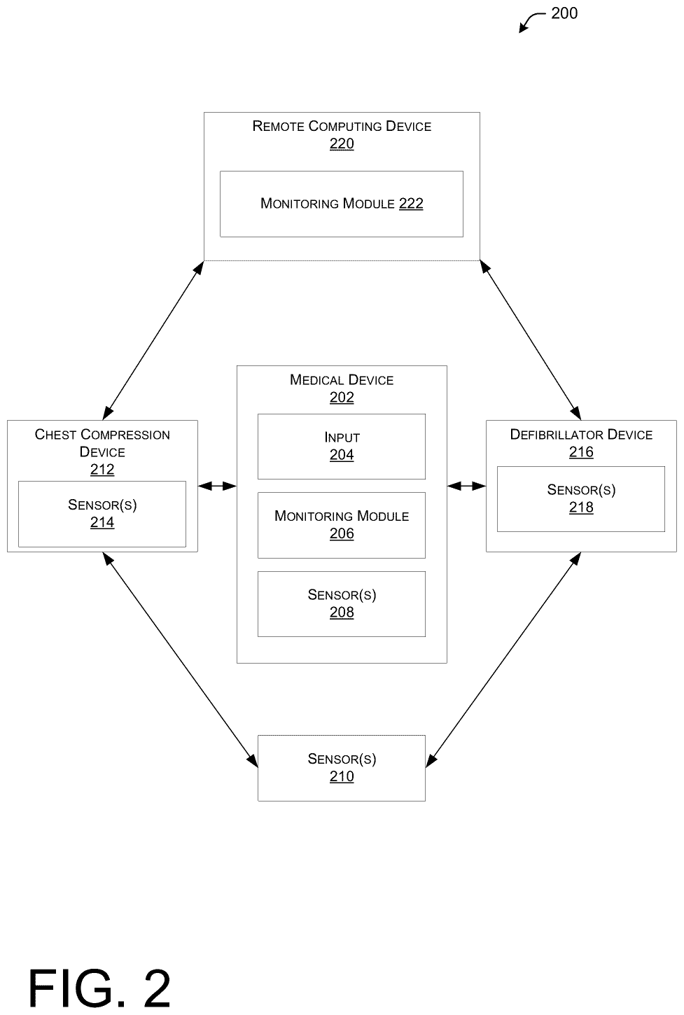

FIG. 2 is an example block diagram of a physiological feedback system with reliability indicators, according to at least one example. The physiological feedback system is provided to assist rescuers, or others, in assessing the physiological state of the patient, and/or assessing the course of the patient's physiologic state over a patient care event, such as a resuscitation effort. The reliability indicators provide valuable indication as the reliability of the data provided by a medical device and/or other rescue device during use without requiring individual monitoring of a separate numerical parameter on a display of the device. For rescuers, the systems and methods can assist with predicting the Return of Spontaneous Circulation (ROSC), assessing the hemodynamic stability of the patient who has achieved ROSC for potential decline in the stability, and/or determining eligibility of the patient for more intensive resuscitation procedures that are only considered appropriate for a carefully-selected subset of patients. The described systems and methods can allow the rescuer to continuously monitor the physiological state of the patient and the effects of treatments performed thereon. The “whole picture” monitoring provided the rescuers can assist in determining the stability and/or transportability of the patient. Rather than relying on a singular or narrow criterion, the rescuer, and/or others, can be provided a comprehensive patient monitoring/treatment output from which further monitoring and/or treatment decisions can be made. Additionally, this iterative/continuous patient monitoring of the patient's physiological state can assist with assessing potential physiological damage of the patient caused by the event they are experiencing. This damage assessment can also be used to assist with determining further patient treatment and/or monitoring.

In some examples, the system can use regional oximetry, pulse oximetry, and/or capnography data to provide feedback regarding monitoring and/or treatment of a patient. The feedback can include instructions to, and/or can be used to, alter, or otherwise change, the administration of treatment to the patient. For example, of one or more parameters of cardiopulmonary resuscitation (CPR) administration can be altered/changed based on the collected and/or analyzed tissue oximetry and/or capnography data. The tissue oximetry and/or capnography data can be used to derive an index to aid in the monitoring and/or treatment of the patient. Additionally, the tissue oximetry and/or capnography data can be used to determine whether the patient has ROSC and/or predict its likelihood. Further, the treatment and monitoring data can be correlated, such as by using the tissue oximetry and/or capnography data, to monitor the physiological effect of one or more treatments administered to the patient.

FIG. 2 is an example patient treatment/monitoring system 200 that includes a medical device 202 that can be connected to and/or in communication with a sensor(s) 210, other patient monitoring and/or treatment devices/systems such as a chest compression device 212, a defibrillator 216, and/or a remote computing device 220. The medical device 202 can receive patient data from one or more sources and can provide regional tissue oximetry (rSO₂) data—which describes the aggregate oxygenation state of the blood within a certain region of tissue—along with related data, to assist with patient monitoring and/or treatment. The data may also include SpO₂ data from a pulse oximeter. Using the patient data, the medical device 202 can display and/or provide information regarding the patient status. The medical device 202, and/or the remote computing device 220 provide an indication of the reliability of data from the sensor(s) 210 in an intuitive manner that can be easily digested by a user during treatment. The medical device 202 can provide the reliability indication by determining a reliability index for the data and altering a display of the data from the sensor for consumption by the user.

The medical device 202 can include an input 204, a monitoring module 206 and/or a sensor(s) 208. To collect and/or receive patient data, the medical device can include the sensor(s) 208 and/or can communicate with other devices and/or systems, such as the sensor(s) 210, a chest compression device 212, a defibrillator 216, the remote computing device 220 and/or other external devices and/or systems. The collected and/or received patient data can include oximetry data, such as cerebral and/or other tissue oximetry data, and/or can include capnography data among other types of data regarding patient physiological parameters. The oximetry and/or capnography data can be analyzed and/or evaluated by the medical device 202 in correlation with the other received patient data, such as patient physiological and/or treatment data. Additionally, the medical device 202 can receive input 204 from a user and/or an external device/system. The input 204 can include patient treatment and/or observation data. For example, the input 204 can include data regarding medication administration, such as a medication identification, dosage, and/or other administration data and/or can include patient observation data, such as patient gasping, consciousness and/or purposeful movement, that can be provided by a user and/or device/system. Additionally, the input 204 can provide data regarding the weighting and/or importance of one or more of components of the patient data, such as a particular physiological parameter. Such input can be provided by a user, device and/or system. The medical device 202 can determine reliability indices for one or more physiological parameters and display the same, or provide for display, the physiological parameters using a display setting determined based on the reliability indices as discussed herein.

The process of receiving and analyzing patient data and determining reliability indices can be a continuous and iterative process. This allows the analysis and/or evaluation of patient data performed by the medical device 202 to be refined as patient treatment and/or monitoring continues. That is, events throughout the patient monitoring and/or treatment session can be used to refine analysis and/or evaluation of additional collected and/or received patient data to assist with patient monitoring and/or treatment. Using the repeated/continuous and/or iterative analysis and/or evaluation of patient data, the medical device 202 can provide trend data for one or more physiological parameters of the patient. The trend data can assist with and/or guide additional, or further, patient monitoring and/or treatment. Additionally, the trend data can reduce immediate and/or temporary changes to patient physiological parameter data so that decision making by a user, device and/or system can be based, at least in part, on trends in the data rather than solely on a real-time value of the data.

The monitoring module 206 receives and/or collect patient physiologic data, including oximetry and/or capnography data, such as by the sensor(s) 208 and/or 210. The monitoring module 206 may include a processor executing instructions stored on a memory. Oximetry and/or capnography data can be tracked by the medical device 202 during the patient monitoring/treatment session and can provide data regarding the physiological state of the patient. The physiological parameters of oximetry and/or capnography are not only indicative of the patient physiological state, but are also parameters that exhibit correlation with each other. That is, increases in tissue oximetry are often accompanied by increased capnography measurements, as increased tissue oxygenation status during CPR is typically associated with increased blood flow which also causes increases in expired CO₂. This correlation/relationship can allow the two parameters to validate each other and can also assist in validating, evaluating and/or assessing changes in other physiological parameters, such as changes in the patient electrocardiogram, blood pressure and/or other physiological parameters.

The monitoring module 206 determines a reliability index of a physiological parameter such as the capnography or oximetry, or another parameter. The reliability index is a score associated with an accuracy and/or a predicted accuracy of the physiological parameter. The monitoring module 206 also determines a visibility setting for the physiological parameter on a display associated with the medical device 202, such as included with the medical device 202 and/or with the remote computing device 220. The monitoring module 206 further causes the parameter to be displayed using the display setting. The visibility setting reflects a visual clarity of the representation of the physiological parameter on the display.

In some examples, the monitoring module 206 determines the reliability index by cross-correlating a first subset of the physiological parameter data with a second subset of the physiological parameter. The cross-correlation is a measure of a similarity of the two subsets and in some examples is performed using a convolution, inner product, correlation matrix, or other such calculations of cross-correlation. In some examples, the monitoring module 206 includes a machine learning component trained to output the reliability index in response to input of the physiological parameter and/or sensor data from the sensor(s) 208 and sensor(s) 210 used to derive the physiological parameter.

In some examples, the visibility setting includes a visual sharpness and/or blurriness for the representation of the physiological parameter. The visual sharpness scales proportionally with the reliability index such that a lower reliability index is associated with a lower visual sharpness (e.g., a higher blurriness) and a higher reliability index is associated with a higher visual sharpness (e.g., a lower blurriness). The visibility setting also includes a color of the display, a color of a portion of the display, a color of text or numerical representations within the display, a color of a graphical representation, a brightness level of the display, a brightness of a portion of the display, a brightness of text, a brightness of a line of a graphical representation, a contrast level for the display and/or for a text or graphical item, a text size, a pixilation level (e.g. the degree to which text and/or graphical representations appear pixelated, similar to a blurriness), or other such display setting. In some examples, the visibility setting is associated with an audio signal output by an audio output device as described herein. The audio signal is scaled similar to the visibility setting, for example to scale a volume, pitch, proportion of static within the audio signal, frequency, tone, or other setting of the audio signal output by the audio output device.

The output of the medical device 202 can assist with treatment and/or monitoring of the patient by a user. In an example, a user can be administering cardiopulmonary resuscitation (CPR) to a patient. The output of the medical device 202 can be used to assist with, CPR feedback. The user can receive the CPR feedback and can adjust, and/or receive instructions to adjust, the administration of the CPR to the patient to assist with increasing the efficacy of the administered CPR. While the CPR feedback can include comparison of the administered CPR to a CPR administration model, such as including a range of preferred chest compression depth and/or rate, the CPR feedback provided by, or based on, the output of the medical device 202 also compares, or accounts, for the effectiveness of the administered CPR by monitoring and/or analyzing trends in the collected/received patient data. The reliability of such data and trends is presented in an intuitive manner by adjusting the display settings as discussed herein for ease of understanding while using the medical device 202, for example while performing CPR.

In addition to receiving information, such as patient data, from the one or more external users, devices and/or systems, the medical device 202 can also provide information, such as the output, to the external users, devices and/or systems. In the example shown in FIG. 2, the medical device 202 can communicate with other treatment/monitoring devices/systems, such as the chest compression device 212 and/or defibrillator 216. The chest compression device 212 and/or the defibrillator 216 can receive the output, or other information/instructions, from the medical device 202, which can alter the monitoring and/or treatment of the patient based on the received output, or other information/instructions.

The chest compression device 212 can administer chest compressions, such as part of a CPR treatment, to a patient and can include sensors 214, such as for monitoring the administration of compressions by the chest compression device 212 and/or one or more physiological parameters of the patient. Additionally, or alternatively, the chest compression device 212 can receive patient physiological and/or other data from the optional sensor(s) 210. The chest compressions administered by the chest compression device 212 can have specific characteristics, including a depth of compression, a velocity of the administered compression, and/or a rate at which compressions are administered. Further, the chest compression device 212 can also administer active decompressions by actively lifting the patient's chest. The sensors 214 can transmit information to the medical device regarding the operation of the chest compression device 212 and/or the physiological state of the patient. The chest compression device 212 can also receive instructions from the medical device 202 to alter the administration of compressions by the chest compression device 212. The received instructions can automatically cause the chest compression device 212, and/or require at least some user input to cause the chest compression device 212, to alter the administration of compressions and/or active decompressions to the patient.

The chest compression device 212 can also communicate with one or more remote computing devices 220. The communication can include sending and receiving of information and/or instructions. That is, the remote computing device 220 can receive data from the chest compression device 212, such as sensor 214 data, and/or can transmit instructions to the chest compression device 212, such as to alter administration of treatment by the chest compression device 212.

The defibrillator 216 can administer defibrillation therapy, such as electrical shocks, to the patient and can include a sensor(s) 218, such as for monitoring one or more physiological parameters of the patient. Additionally, or alternatively, the defibrillator can receive patient physiological and/or other data from the optional sensor(s) 210. The defibrillator 216 can transmit and/or receive information with the medical device 202. The information transmitted to the medical device 202 can include patient physiological information and/or patient treatment information, such as information regarding administered defibrillation therapies. The information received by the defibrillator 216 can include the output, and/or instructions, from the medical device 202. The output, and/or the instructions, from the medical device 202 can alter patient monitoring and/or treatment by the defibrillator 216. In an example, the medical device 202 can track the administration of defibrillation shocks to the patient and the resulting effects of the administered treatment. The output of the medical device 202 can be used by, and/or include instructions for, the defibrillator 216 to provide an altered, or different, treatment to the patient based on the previous treatment and/or the physiological state trends of the patient.

The defibrillator 216 can also communicate with the one or more remote computing devices 220, which includes the computing device 120 of FIG. 1. The communications can include sending and receiving of information and/or instructions. That is, the remote computing device 220 can receive data from the defibrillator 216, such as sensor(s) 218 data, and/or can transmit instructions to the defibrillator, such as to alter the administration of treatment by the defibrillator 216.

The remote computing device 220 can communicate with the medical device 202 and/or other devices and/or systems that are used in monitoring and/or treating the patient. The medical device 202 and/or other devices and/or systems can receive data and/or instructions from the remote computing device and can provide data, such as patient physiological and/or treatment data, to the remote computing device 220. The remote computing device 220 can also provide additional patient information, such as previous treatment and/or monitoring history to other users, devices and/or systems, such as the medical device 202. The remote computing device 220 can be a user device and/or system external from the medical device 202, such as a mobile computing device that includes a monitoring module 222 similar to the monitoring module 206 of the medical device 202 that is configured to determine reliability indices for physiological parameters and/or sensor data from the data received from the sensors and/or devices within the system 200.

The remote computing device 220 can be a portable device, such as a tablet, that can be connected to the medical device 202 and/or other devices/systems, such as by an integrated patient monitoring and/or treatment network. The remote computing device 220 can receive, and/or obtain, patient information, such as from the medical device 202, and can provide an interface for a user to interact with one or more of the connected devices and/or systems, such as the medical device 202. The user can interact with the remote computing device 220 to evaluate the patient data, to provide instructions regarding patient treatment and/or monitoring and/or for other functions and/or features. Additionally, the remote computing device 220, and/or other remote computing devices, can provide, or assist, with analysis of the patient data, such as physiological trend(s) analysis, reliability analysis, and other such analysis that can be provided to the medical device 202 and/or other users, devices and/or systems.

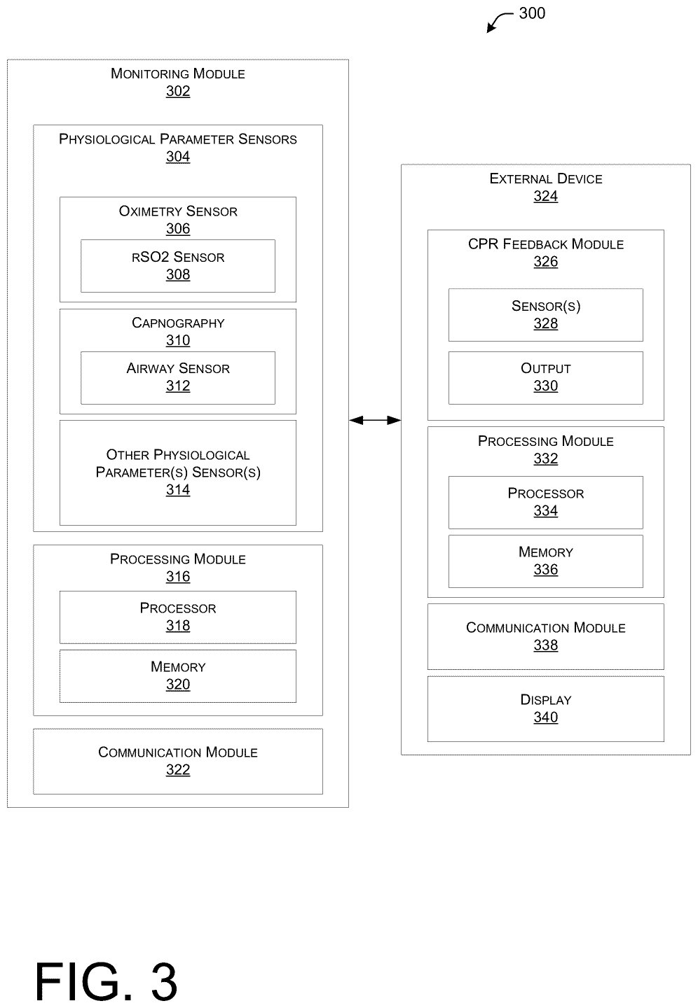

FIG. 3 is an example block diagram of a system 300 for monitoring physiological parameters and providing indication of reliability, according to at least one example. The system 300 includes a monitoring module 302 and an external device 324. The monitoring module 302 may include a processor executing instructions stored on a memory. In some examples, the monitoring module 302 may include a processor 318 and memory 320. The processor 318 can analyze and/or evaluate data, such as received from the physiological parameter sensors 304 and/or the communication module 322, and/or control one or more functions and/or features of the monitoring module 302. The processor 318 can additionally be configured to process instructions stored on the memory 320 to cause the processor to determine reliability index information for the physiological parameters and/or sensor data associated with the sensors 304. The memory 320 can store data, such as received from the physiological parameter sensors 304 and/or the communication module 322, and/or instructions and/or processes for the processor 318 to perform.

The external device 324 can monitor and/or treat a patient while in communication, or connected, with the monitoring module 302. The monitoring module 302 can sense and/or receive capnography and/or oximetry data (or other sensor data) of the patient to monitor and/or assess an aspect of the physiologic state of the patient. The monitoring module 302 and/or the external device 324 can use the physiologic state information to assess treatment efficacy, such as cardiopulmonary resuscitation (CPR) effectiveness. In response to the assessment, the monitoring module 302 and/or the external device 324 can advise, or instruct, on further treatment and/or modification, or alteration, of treatment being administered, such as CPR.

In addition to assessing CPR effectiveness, the physiologic state information of the patient can also be used to assess brain function based on the cerebral tissue oxygenation. During cardiac arrest, blood flow is ineffective and oxygen deprivation of various tissues results due to the lack of blood flow. CPR mechanically forces blood flow so that oxygen deprivation is slowed. Brain and nervous tissue is susceptible to damage due to oxygen deprivation, so cerebral tissue oxygenation information can also be used to assess the magnitude of oxygen deprivation in the brain. Trends in cerebral tissue oxygenation over time can also be used to assess the likelihood and/or magnitude of damage to the brain, and such information can in turn be used in decision-making regarding additional treatments and resuscitation efforts.

The monitoring module 302 can include physiological parameter sensors 304, a processing module 316 and a communication module 322. The physiological parameter sensors 304 can be connected to and/or communicate with the monitoring module 302. Alternatively, the sensor data can be provided to the monitoring module 302 from one or more external devices and/or systems. The physiological parameter sensors 304 can include one or more oximetry sensors 306, one or more capnography sensors 310, and one or more other physiological parameter(s) sensors 314, in some examples. The oximetry sensors 306 can provide sensor data regarding tissue and/or blood oxygenation levels of a patient which one or more oximetry sensors 306 are monitoring. The capnography sensors 310 can provide sensor data regarding the expired CO₂ levels of the patient. The other physiological parameter(s) sensors 314 can provide sensor data regarding other physiological parameters, such as ECG data, non-invasive blood pressure data, pulse oximetry data and/or other data regarding other physiological parameters.

The oximetry sensors 306 can include regional tissue oxygenation (rSO₂) sensors 308 and/or SPO₂ that can be placed on the patient to monitor oxygenation of the tissues underneath the sensors. One or more of the rSO₂ sensors 308 can be placed on the patient's head, such as on the patient's forehead, to monitor cerebral tissue oxygenation, for example. The rSO₂ sensors 308 can be light-based sensors that include one or more light emitters and detectors. The light emitters of the rSO₂ sensors 308 can emit Near Infrared light having various light characteristics, such as one or more frequencies and/or wavelengths. The emission of Near Infrared light having multiple wavelengths can be used to sense oxygenation of blood at various depths beneath the rSO₂ sensor 308. Near Infra-Red Spectrometry (NIRS) can be used to calculate the oxygenation level of the blood in tissues under the rSO₂ sensor 308. The NIRS processing can be performed by the monitoring module 302, and/or by an external device and/or system, to determine the blood/tissue oxygenation data. Additionally, the NIRS processing can provide a broad measure of blood oxygenation levels by providing oxygenation data that is a combination of venous and arterial blood oxygenations. The oximetry data, such as provided by the rSO₂ sensor 308 and processing of data therefrom, can provide a measure of the patient's oxygenation state and/or the balance between oxygenated and deoxygenated blood in the tissue being measured by the sensor.

The capnography sensors 310 measure fraction or partial pressure of CO₂ in gases in the airway, and from that airway CO₂ signal end-tidal CO₂ (EtCO₂) can be calculated. An airway sensor 312 can monitor the CO₂ expelled from the patient which can provide an indication of the patient's CO₂ levels and, since blood flow is the primary means of transport of CO₂ from the lungs, an indication of the amount of blood flow occurring in the patient.

The processing module 316 can include a processor 318 and memory 320. The processor 318 can analyze and/or evaluate data, such as received from the physiological parameter sensors 304 and/or the communication module 322, and/or control one or more functions and/or features of the monitoring module 302. The processor 318 can additionally be configured to process instructions stored on the memory 320 to cause the processor to determine reliability index information for the physiological parameters and/or sensor data associated with the sensors 304. The memory 320 can store data, such as received from the physiological parameter sensors 304 and/or the communication module 322, and/or instructions and/or processes for the processor 318 to perform.

The communication module 322 can communicate with external devices and/or systems, such as the external device 324, using one or more communication protocols and/or connections, such as Wi-Fi, the Internet, Bluetooth® and/or other protocols and/or connections. Data can be transmitted from and/or received to the monitoring module 302 via the communication module 322. For example, the communication module 322 can receive physiological parameter data and/or treatment data from the external device 324 and can transmit a tissue oxygenation value and/or treatment instructions to the external device 324.

The external device 324 can include a CPR feedback module 326, a processing module 332 and a communication module 338. The external device 324 can monitor the physiological state of the patient and/or monitor patient treatment and provide instructions for additional treatment and/or modification of the current patient treatment. Monitoring a patient's physiological state and/or treatment can include collecting physiological parameter data from the patient and/or data from the administration of treatment to the patient.

The CPR feedback module 326 can include a sensor(s) 328 to monitor one or more parameters of CPR administration, such as compression rate and/or depth, and an output 330. The sensor 328 can be connected to and/or in communication with the CPR feedback module 326, to provide data regarding one or more CPR parameters. Alternatively, the CPR parameter data can be supplied to the external device 324 by another device and/or system that generates CPR parameter data. The output 330 can provide information to a user regarding the administered CPR, such as feedback, including an assessment of the administered CPR and/or instructions to alter one or more parameters of the administered CPR. The output can communicate this information in a visual and/or audible format, such as by a display screen and/or a speaker. The user can interpret the provided visual and/or audible output 330 to initiate and/or modify treatment of the patient.

The processing module 332 includes a processor 334 and memory 336. The processor 334 can analyze and/or evaluate data, such as the tissue oxygenation value received from the monitoring module 302, and/or control one or more functions and/or features of the external device 324. The processor 334 can additionally be configured to process instructions stored on the memory 336 to cause the processor to determine reliability index information for the physiological parameters and/or sensor data associated with the sensors 328. The memory 336 can store data, such as received from the CPR feedback module 326 and/or the communication module 338, and/or instructions and/or processes for the processor 334 to perform.

The communication module 280 can communicate with external devices and/or systems, using one or more communication protocols and/or connections, such as Wi-Fi, the Internet, Bluetooth® and/or other protocols and/or connections. Data can be transmitted from and/or received to the external device 324 via the communication module 280. For example, the communication module 338 can receive tissue oxygenation data and/or treatment from the monitoring module 302 and can transmit CPR parameter data to the monitoring module 302.

Though described with respect to oximetry, capnography, and CPR for the system 300, other medical device systems implement a monitoring module 302 configured to receive the other physiological parameters 314 and determine reliability index information associated therewith for display on a device associated with the external device.

In an embodiment, the monitoring module 302 can monitor tissue oxygenation levels of the patient using the oximetry sensor data 306, and/or levels of CO₂ expired by the patient using capnography sensor 310 data. From the collected oxygenation data, the monitoring module 302 can calculate a tissue oxygenation value. From the collected capnography data, the monitoring module can calculate EtCO₂, providing an indirect assessment of pulmonary blood flow. The external device 324 can collect CPR parameter data, such as compression depth and rate, and can transmit the collected CPR parameter data to the monitoring module 302. The monitoring module 302 can use the received CPR parameter data, and/or the calculated tissue oxygenation value, and/or the EtCO₂ level, to determine an effectiveness, or feedback, of the CPR being administered. In response to that determination, the monitoring module 302 can provide instructions to the external device 324 to cause the administrator of the CPR to alter one or more of the CPR parameters and/or can provide the CPR feedback data to the external device 324 for output and/or CPR instruction/alteration determination and output. In this manner, CPR effectiveness can be determined based on tissue oxygenation data, and/or airway CO₂ data, and one or more parameters of CPR administration can be altered in response to, and/or based on, the tissue oxygenation data. Alteration of the CPR administration can be done to increase or redirect blood flow and thereby assist with increasing tissue oxygenation levels which can assist with preventing damage, such as due to hypoxia, and/or can improve the probability of achieving ROSC. In a further, or alternate, embodiment, the external device 324 can be a mechanical CPR device, such as a chest compression machine (chest compression device), and the operation of the mechanical CPR device can be automatically altered, or altered at the discretion of the rescuer, based on the tissue oxygenation data and/or the airway CO₂ data.

Furthermore, during treatment using the system 300, the monitoring module determines reliability index information and/or determine display settings for displaying the data from the sensors 304 and/or from the CPR feedback module 326. For example, the capnography sensor 310 detects data associated with a respiration rate of the patient. The capnography sensor 310 conveys data associated with the capnography sensor and the respiration rate sensor to the computing device monitoring module 302 and/or the external device 324 where a physiological parameter of the patient is determined using the data from the capnography sensor 310. For instance, the monitoring module 302 determines an end-tidal carbon dioxide parameter for the patient by analyzing the capnography sensor 310 data. The end-tidal carbon dioxide is displayed on a display 340 of the external device 324. The external device 324 further causes the display 340 to show the respiration rate data.

In the example, the respiration rate drops below a threshold level for a threshold period of time, which is a result of sensor error or a result of slowing or stopping respiration of the patient—very different clinical conditions requiring different actions on different timeframes that need to be distinguished to determine if intervention with the patient is warranted. For example, when the respiration rate of the patient, as detected, drops below five breaths per minute over a threshold period of time of ten to thirty seconds or more. In some examples, other thresholds, such as higher or lower breaths per minute over greater or shorter periods of time is introduced as well. The drop in the respiration rate is associated with a drop in a reliability score, as determined by the monitoring module 302, for the end-tidal carbon dioxide, or other parameters, as the drop in the respiration rate is a sign of unreliable data. Accordingly, the external device 324 causes the display 340 to output a representation of the capnograph data, rather than the derived end-tidal carbon dioxide value.

In some examples, the end-tidal carbon dioxide value is removed from the display 340 in response to the monitoring module 302 determining the reliability of the data is below a threshold value for a threshold period of time. For example, the end-tidal carbon dioxide is removed from the display 340 and replaced with a representation of the capnograph data from the capnography sensor 310. The capnograph data is displayed as a chart, graph, numerical value, or other representation, with an indicator that the display 340 is presenting capnograph data rather than end-tidal carbon dioxide. In some examples, the display of the end-tidal carbon dioxide and the capnograph data is presented on the display 340 in different types of representations (e.g., a numerical value versus a graphical representation) to enable a user to rapidly identify the type of data displayed. In such examples, the capnograph data is presented over a period of time, rather than at an instant point in time, with the capnograph representing data detected at a present time and a previous time while the end-tidal carbon dioxide is presented as an instantaneous numerical value associated with a present value.

In the example, the display 340 additionally has a feature altered to reflect the change in data presented on the display 340. For instance, when the capnograph data is presented on the display rather than the end-tidal carbon dioxide, a color of a portion of the display 340 associated with the representation changes, such as to a bright or highly visible color such as red or yellow to draw attention to the change in the displayed data. In some examples, the brightness of the display 340 is also changed, across the entire display 340, such as to increase a brightness when the capnograph data is presented, and/or by changing a label of data on the display 340 to label the capnograph data as such.

In some examples, the external device 324 further causes an audio output. The audio output is altered in response to presenting the capnograph on the display 340. For instance, the audio signal includes a tone when the capnograph is initially presented. In some examples, the audio signal is a tone that changes in pitch and/or volume in response to displaying the capnograph.

The monitoring module 302 determines a reliability index of a physiological parameter such as the capnography or oximetry data, or another parameter. The reliability index is a score associated with an accuracy and/or a predicted accuracy of the physiological parameter. The monitoring module 302 also determines a visibility setting for the physiological parameter on a display 340 associated with the system 300. The monitoring module 302 further causes the parameter to be displayed using the display setting. The visibility setting reflects a visual clarity of the representation of the physiological parameter on the display.

In some examples, the monitoring module 302 determines the reliability index by cross-correlating a first subset of the physiological parameter data with a second subset of the physiological parameter. The cross-correlation is a measure of a similarity of the two subsets and in some examples is performed using a convolution, inner product, correlation matrix, or other such calculations of cross-correlation. In some examples, the monitoring module 302 includes a machine learning component trained to output the reliability index in response to input of the physiological parameter and/or sensor data from the oximetry sensor 306 and/or capnography sensor 310, and/or the other physiological parameter 314 used to derive the physiological parameter.

In some examples, the visibility setting includes a visual sharpness and/or blurriness for the representation of the physiological parameter. The visual sharpness scales proportionally with the reliability index such that a lower reliability index is associated with a lower visual sharpness (e.g., a higher blurriness) and a higher reliability index is associated with a higher visual sharpness (e.g., a lower blurriness). The visibility setting also includes a color of the display, a color of a portion of the display, a color of text or numerical representations within the display, a color of a graphical representation, a brightness level of the display, a brightness of a portion of the display, a brightness of text, a brightness of a line of a graphical representation, a contrast level for the display and/or for a text or graphical item, a text size, a pixilation level (e.g. the degree to which text and/or graphical representations appear pixelated, similar to a blurriness), or other such display setting. In some examples, the visibility setting is associated with an audio signal output by an audio output device as described herein. The audio signal is scaled similar to the visibility setting, for example to scale a volume, pitch, proportion of static within the audio signal, frequency, tone, or other setting of the audio signal output by the audio output device.

While the monitoring module 302 and the external device 324, such as a defibrillator, patient monitor, monitor/defibrillator, mechanical CPR device and/or other medical treatment and/or monitoring device, are shown as separate elements, one or more features and/or functionality of one or more of the monitoring module 302 and the external device 324 can be combined and/or integrated with the other and/or another device.

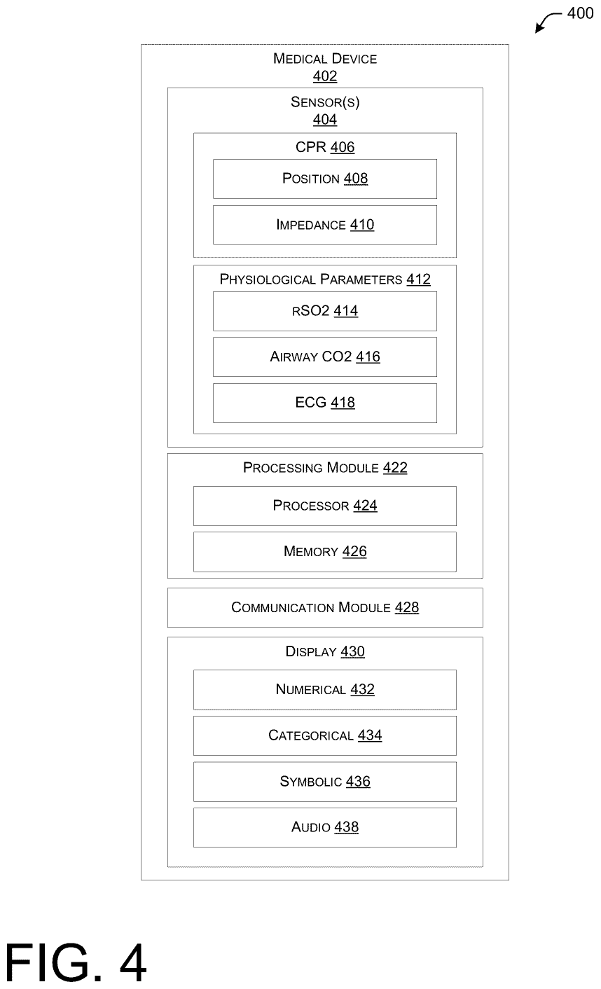

FIG. 4 is an example block diagram of a physiological monitoring system 400 with a display for outputting the parameter and reliability of the parameter, according to at least one example. The system 400 includes a medical device 402. The medical device 402 can include one or more sensors 404, a processing module 422, a communication module 428 and a display 430. The one or more sensors 404 can be coupled to a patient to receive/sense data regarding the patient, such as one or more physiological characteristics/parameters of the patient. Alternatively, or additionally, the medical device 402 can receive data regarding the patient, such as physiological parameter data, from an external device, system and/or user. The medical device 402 can analyze, process and/or evaluate the sensed/received data to provide physiological feedback regarding the patient, such as treatment metrics and/or other feedback to guide treatment and/or monitoring of the patient.

The sensor(s) 404 can be placed on, or near, the patient and connected to, or in communication with, the medical device 402 to provide sensor data indicative of one or more physiological parameters of the patient, a physiological condition of the patient, treatment administered to the patient and/or other data regarding the patient. In the example of FIG. 4, the sensors 404 include one or more cardiopulmonary resuscitation (CPR) sensors 406 and/or one or more physiological parameter sensors 412. The CPR sensors 406 can provide data regarding the administration of CPR, or lack thereof. The data can include measurements of one or more CPR variables, characteristics of the CPR administration and/or other data regarding the administration of CPR. The physiological parameter sensors 412 can provide data regarding one or more physiological parameters of the patient.

The CPR sensors 406 can be placed on and/or near the patient and can include a position sensor 408, an impedance sensor 410 and/or other sensors to measure, monitor and/or assess the administration of CPR to a patient. The position sensor 408 can include one or more elements that are placed on the patient to which CPR is being administered, on the person of a user administering CPR to the patient, and/or placed proximal the patient, such as above the patient, on a surface near the patient, beneath the patient and/or other patient adjacent locations. One or more signals can be generated by the position sensor 408 that can be indicative of one or more parameters/characteristics of the administered CPR, such as a rate of compressions, the depth of compressions, the number of compressions and/or other parameters/characteristics related to the administration of CPR to the patient. The impedance sensor 410 can measure a transthoracic impedance of the patient, such as by a pair of electrodes placed on the patient, and the measured data can be indicative of CPR administration since the transthoracic impedance of the patient changes in response to administered compressions.

The physiological parameter sensors 412 can include a regional tissue oximetry (rSO₂) sensor 414, such as to measure cerebral tissue oximetry, an airway CO₂ sensor 416, from which end-tidal CO₂ (EtCO₂) can be calculated, an electrocardiogram (ECG) sensor 418 and/or other physiological parameter sensors. The EtCO₂ refers to measurement of how much carbon dioxide a person is exhaling. A capnograph and/or capnography data refers to breath-by-breath analysis and recording of ventilatory status. The sensor signals generated by the rSO₂ sensor 414 and/or the airway CO₂ sensor 416 can be indicative of the level of blood flow in the patient. Such data can be used to assess and/or analyze the physiological state of the patient. Additionally, such physiological parameter data can provide information regarding potential future patient physiological states, provide information for treatments and/or interventions, and/or other information for use in monitoring and/or treating the patient. Further, the collected physiologic and/or patient data can be used for pattern, and/or other, analysis to aid in developing and/or refining the plan for ongoing patient treatment, monitoring and/or assessment.

The ECG sensor 418 can include two or more electrodes that are placed on the patient and provide sensed data to the medical device 402.

Reception of sensed data, from the sensors 404, by the medical device 402 can be via wired and/or wireless connection(s). In an embodiment in which the transmission of data is via a wireless connection, the sensed data and/or the connection can be encrypted/secured to protect the integrity of the transmitted sensor data. Additionally, the sensed data can be communicated to the medical device 402 from one or more other devices and/or systems that monitor/sense physiological parameter data of the patient.

The processing module 422 can include a processor 424 and memory 426. The processing module 422 can control one or more functions and/or features of the medical device 402. Additionally, the processing module can receive various data/information, such as from the sensors 404 and/or a user, device and/or system, for collection and/or analysis. The collection and/or analysis of data by the processing module 422 can assist with patient assessment, treatment and/or monitoring. Further, such data can also be processed for multiple patients to determine trends and/or patterns that can assist with future patient assessment, monitoring and/or treatment. The processing module 422 can also collect and store information regarding patient instances, such as assessment, monitoring and/or treatment data, that can be transmitted, or provided, to a user, device and/or system upon conclusion of the patient instance.

The processing module 422 is configured to perform the functions of the monitoring module 302 of FIG. 3, and therefore is configured to determine reliability information associated with the sensed and/or determined data, and subsequently determine display and/or output settings for the display 430 based on the reliability information as described herein.

The communication module 428 can transmit and/or receive information from/to the medical device 402 and one or more external devices and/or systems. The communication module 428 can communicate with the one or more external devices and/or systems via one or more communication protocols and/or connections, such as Bluetooth®, Wi-Fi, the Internet and/or other communication protocols and/or connections. Communications to and/or from the communication module 428 can be via a secure communication channel and/or can be encrypted, to preserve the integrity, or security, of the communications. In an example, one or more of the sensors 404 can be part of an external device/system that transmits sensed data to the medical device 402 via the communication module 428.

The display 430 can provide information in a visual format to a user, device and/or system. To provide the information, the display 430 can include one or more screens, lights and/or other visual indicators, to display, or provide, information in a visual format. The display 430, or portion thereof, can also be configurable allowing the format and/or other characteristics of the display to be altered, such as in response to the user and/or by the processing module 422. The configurability of the display 430, or portion thereof, can allow the display 430 to provide relevant information in a more accessible manner, such as by highlighting priority information more than other information displayed by the display 430. For example, the reliability index information determined by the processing module 422 is used to determine display settings that highlight or de-emphasize particular data and information based on the reliability index associated therewith.

Example visual formats the display 430, or portion thereof, can display information in a numerical format 432, a categorical format 434 and/or a symbolic format 436. The numerical format 432 can include displaying numerical values, such as measurements of physiological parameters, on the display 430. The categorical format 434 can include displaying and/or highlighting a category, such as a negative, neutral and/or positive category for a variable like a physiological parameter. The categorical format 434 can also be represented by one or more colors that can change depending on the value of the category. For example, the color green associated with a category can indicate a positive and similarly, yellow can indicate neutral and red can indicate negative. Additionally, or alternatively, the categories of the categorical format can be displayed as variable indicators, such as a changing bar that can move or expand based on the measurement/value of the category. The variable indicator can also include color associations, such as those previously discussed. The symbolic format 436 can include graphical and/or textual representations of data, such as a text message and/or a graph. The format(s) of the display 430 are presented to aid in the speed and accuracy of determining the information represented, or displayed, thereon. Further, the display 430 can alter the format of information displayed based on the importance of the information and/or the reliability index associated with the information. For example, information that is of lesser importance can be represented categorically 434, such as to reduce the area for the display of such information, the display format can change if the information becomes more relevant or important, such as to a numerical 432 and/or larger format to highlight the importance of the information and/or that the importance of such information has changed from a previous state. Again, such functionality assists with presenting the relevant information in a format that assists with the efficient and accuracy determination of the displayed information.

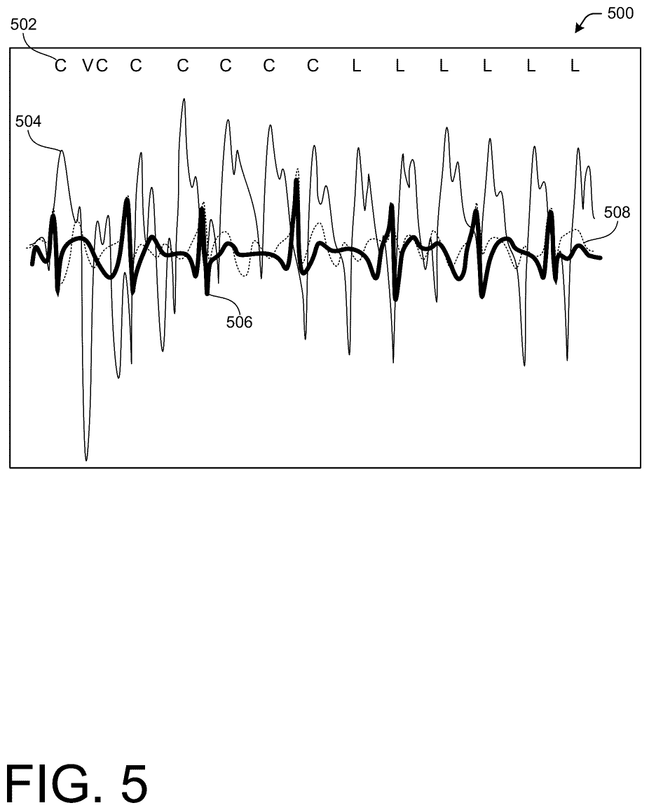

FIG. 5 illustrates an example chart 500 showing information associated with automated or manual rhythm interpretation while chest compressions are ongoing, according to at least one example. FIG. 5 shows an illustration of various different approaches to showing information pertinent to automated or manual rhythm interpretation while chest compressions are ongoing. The display of the chart 500 includes symbols/letters 502 to indicate the detection of events such as manual chest compressions, LUCAS chest compressions, ventilations, QRS sense events, etc. Waveforms are displayed in a way that allows easy recognition of the primary waveform of interest (e.g., filtered ECG 506 with artifact from chest compressions removed) but with a closer look, additional waveforms will be visible due to the display settings (unfiltered ECG 508, transthoracic impedance 504, etc.)

The use of different display settings for the filtered ECG 506 ensures that the filtered signal is highly visible. In some examples, as described herein, the filtered ECG signal 506 has a reliability index associated therewith. The filtered ECG 506 is shown in a bold and/or bright and therefore highly visible line type when the reliability index is high, indicating a high degree of reliability. In contrast, when the reliability index is relatively low, the line type for the filtered ECG 506 is thinner or change to a less prominent color. In some examples, the waveforms coincide on the same channel or be offset into multiple channels. In some examples, when the reliability index for the filtered ECG 506 is below a threshold level for a threshold period of time, the filtered ECG 506 is removed from the chart 500. In addition, the device displaying the chart 500 enables a user to toggle on or off the additional information that is displayed.

In one example, the user can toggle each piece of information or waveform, in another example all the information is toggled at once, in another example both options are present. Each waveform can be adjusted with color, line thickness, brightness, transparency, line style, etc. to create the appropriate visual effects. The line properties of the waveform changes over time in some examples to illustrate a change such as transition from manual chest compressions to LUCAS chest compressions.

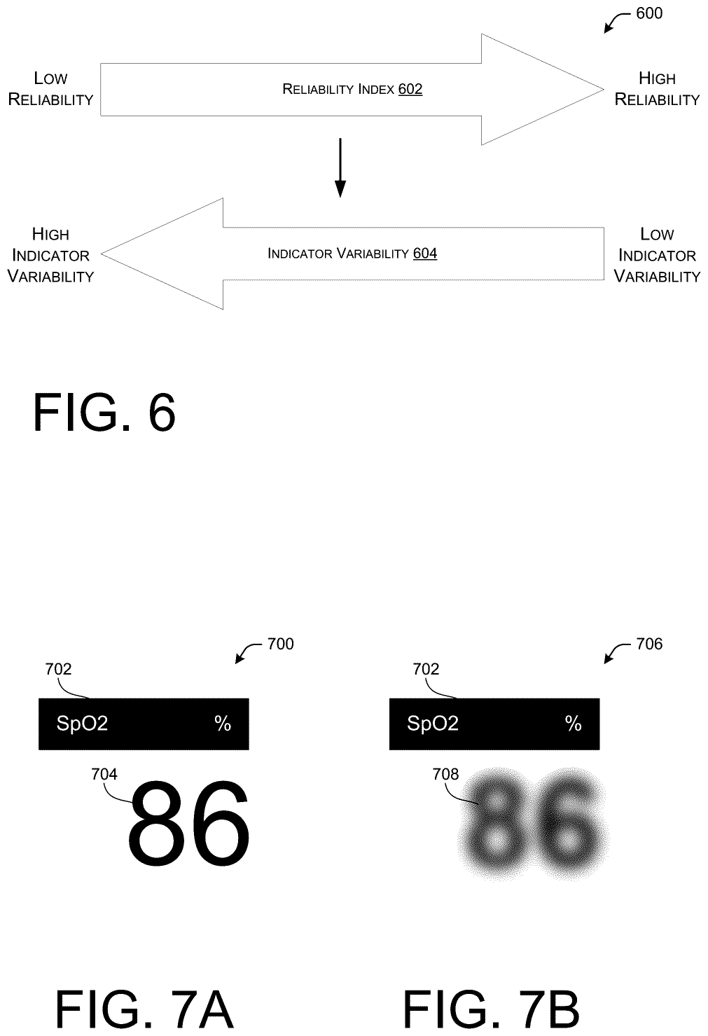

FIG. 6 illustrates a representative scale 600 indicating association between reliability of a physiological parameter and variability in the reliability indicator, according to at least one example. In the scale 600 of FIG. 6, a reliability index 602 is represented as spanning from a low reliability to a high reliability that is associated with one or more signals, either received directly from a sensor or derived from sensor data, as discussed herein. The reliability index, in some examples, includes a reliability score that represents a score between zero and one or between zero and one hundred that reflects a level of reliability in the accuracy of the data. In the scale 600, an indicator variability is shown to vary inversely with the reliability index, with an increase in the reliability index associated with a decrease in indicator variability.

For example, feedback on reliability is provided by blurring the measured number on the display, as discussed with respect to FIGS. 7A and 7B when a reliability index falls below a threshold and/or scales proportionally with the reliability index. In some examples, additional indicators is used to communicate the reliability of one or more vital signs. In one example the vital sign is tied to an audio cue, for example, heart rate is often tied to an audio beep where the beeps are produced each time a QRS is detected (e.g., for ECG) or each time a heartbeat is detected (e.g., for plethysmography).

In one example, when heart rate is measured via plethysmography the pitch of the audio beep can be tied to the saturation level (e.g., a higher pitch for higher saturation). Thus, reliability feedback for plethysmographically or ECG-based heart rates is indicated by modifying the audio cue. In one example, white noise is added at various volume levels based on the reliability index for that vital sign. With respect to the scale 600, as the reliability index 602 increases, the volume of the white noise, the indicator variability 604, decreases. In another example, the white noise remains at a consistent level, but the intensity of the beep associated with the heart rate increases as the reliability index increases; and above a threshold level of reliability the white noise is turned off.

In some examples, instead of adjusting a volume of the white noise, the frequency profile is changed. In one example, when no QRS or heat beat detections occur the audio cue transitions to a background/low level of white noise that is consistent. In another example, the modified cue includes a series of short sounds such as clicks, or fluctuating pitches, or any other audio cue modulation.

In some examples, visual display setting is adjusted as discussed herein. For instance, in one example, the waveform representing a physiological parameter is blurred instead of and/or in addition to a numerical indicator of the vital sign. In another example, the color of the waveform and/or vital sign numerical indicator is modified by, for example, using green for high reliability measurements and red for low reliability measurements. In yet another example, the characteristic of the waveform and or vital sign number is modified according to the reliability index. For example, as the reliability index decreases the waveform and/or numerical representation transitions from solid lines to increasingly sparse dotted or dashed lines. In yet another example, the numerical representation or lines of the waveform is made more faint and/or transparent as the reliability index decreases. In some examples, the display settings includes decreases in contrast between the indicator and the background on the display, line thickness, line color, line brightness, and other such variations in display settings.

In some examples, the reliability index is directly displayed near the vital sign or waveform as a percentage (100% as highly reliable and 0% and not reliable). All of the approaches discussed herein for displaying the reliability index for particular parameters could be used alone or in any combination.

FIG. 7A illustrates an example display 700 of an oxygen saturation value having a first reliability of the physiological data, according to at least one example. In some examples, a pulse oximeter, either as a standalone device and/or as part of a larger system, displays an oxygen saturation reading regardless of the accuracy and/or reliability of the reading. In some examples, a user observes other data, such as a plethysmograph waveform to observe if variations in the oximeter data is related to arterial blood pulsing into a tissue bed or from some other oscillation. Such observations require time and distract the user from other tasks simply to try and confirm the displayed data.

As shown in FIG. 7A, the display associated with a pulse oximeter includes an SpO₂ reading 702. Within the field, a percentage 704 is displayed representing the measured and/or determined value for the oxygen saturation. The percentage 704 is shown using a display setting based on the reliability index associated with the determined and/or measured oxygen saturation value. The reliability is determined by cross-correlating a first subset of the physiological parameter data with a second subset of the physiological parameter. The cross-correlation is a measure of a similarity of the two subsets and in some examples is performed using a convolution, inner product, correlation matrix, or other such calculations of cross-correlation. In some examples, the reliability index is determined by using a machine learning component trained to output the reliability index in response to input of the physiological parameter and/or sensor data from the pulse oximeter used to derive the physiological parameter.

FIG. 7B illustrates an example display 706 of an oxygen saturation value having a second reliability of the physiological data, according to at least one example. The percentage 708 has a second reliability index, determined as described herein. In the example of 7B, the reliability index of the percentage 708 is below a threshold value, resulting in blurring of the displayed value. In some examples, as described herein, the displayed value is blurred according to the reliability index, based on a scaling factor. In some examples the amount of blurring corresponds to the value of the reliability index. In some examples the blurring is binary, with the value blurred if the reliability is below a threshold value, in some examples for a threshold period of time. Upon increase of the reliability index, the percentage 708 is displayed with increased sharpness, decreased blurriness, such as shown in FIG. 7A.

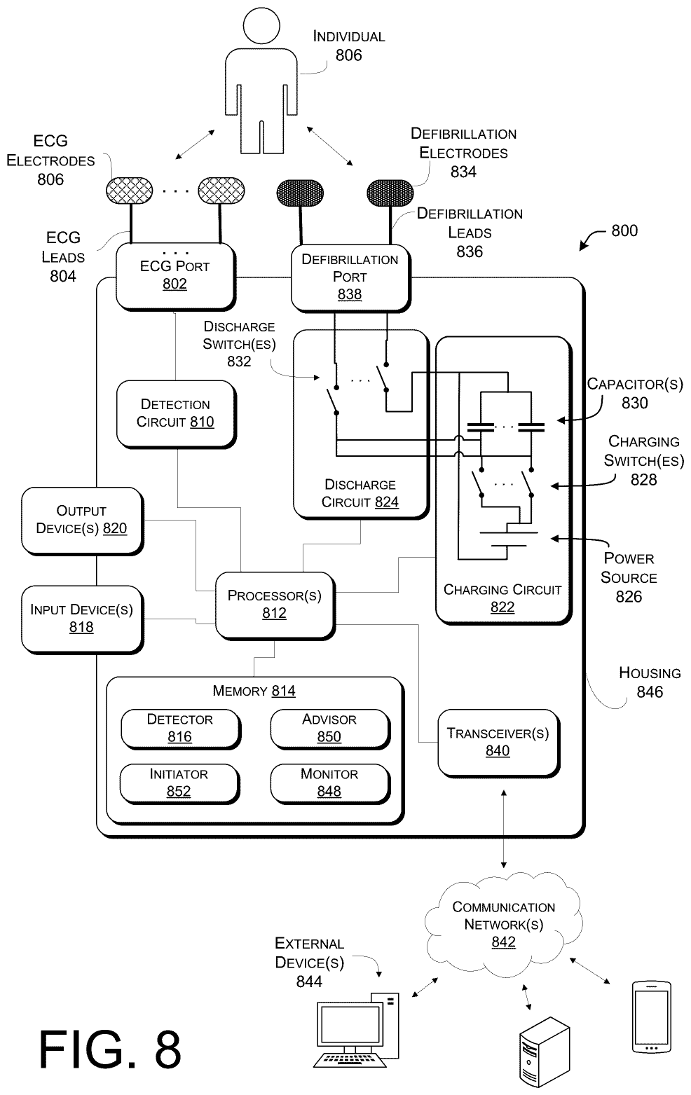

FIG. 8 illustrates an example of an external defibrillator 800 configured to perform various functions described herein. For example, the external defibrillator 800 is the external device 106 described above with reference to FIG. 1 and/or the medical device 202 described above with respect to FIG. 2.

The external defibrillator 800 includes an electrocardiogram (ECG) port 802 connected to multiple ECG leads 804. In some cases, the ECG leads 804 are removeable from the ECG port 802. For instance, the ECG leads 804 are plugged into the ECG port 802. The ECG leads 804 are connected to ECG electrodes 806, respectively. In various implementations, the ECG electrodes 806 are disposed on different locations on an individual 808. A detection circuit 810 is configured to detect relative voltages between the ECG electrodes 806. These voltages are indicative of the electrical activity of the heart of the individual 808.

In various implementations, the ECG electrodes 806 are in contact with the different locations on the skin of the individual 808. In some examples, a first one of the ECG electrodes 806 is placed on the skin between the heart and right arm of the individual 808, a second one of the ECG electrodes 806 is placed on the skin between the heart and left arm of the individual 808, and a third one of the ECG electrodes 806 is placed on the skin between the heart and a leg (either the left leg or the right leg) of the individual 808. In these examples, the detection circuit 810 is configured to measure the relative voltages between the first, second, and third ECG electrodes 806. Respective pairings of the ECG electrodes 806 are referred to as “leads,” and the voltages between the pairs of ECG electrodes 806 are known as “lead voltages.” In some examples, more than three ECG electrodes 806 are included, such that 5-lead or 12-lead ECG signals are detected by the detection circuit 810.

The detection circuit 810 includes at least one analog circuit, at least one digital circuit, or a combination thereof. The detection circuit 810 receives the analog electrical signals from the ECG electrodes 806, via the ECG port 802 and the ECG leads 804. In some cases, the detection circuit 810 includes one or more analog filters configured to filter noise and/or artifact from the electrical signals. The detection circuit 810 includes an analog-to-digital (ADC) in various examples. The detection circuit 810 generates a digital signal indicative of the analog electrical signals from the ECG electrodes 806. This digital signal can be referred to as an “ECG signal” or an “ECG.”

In some cases, the detection circuit 810 further detects an electrical impedance between at least one pair of the ECG electrodes 806. For example, the detection circuit 810 includes, or otherwise controls, a power source that applies a known voltage (or current) across a pair of the ECG electrodes 806 and detects a resultant current (or voltage) between the pair of the ECG electrodes 806. The impedance is generated based on the applied signal (voltage or current) and the resultant signal (current or voltage). In various cases, the impedance corresponds to respiration of the individual 808, chest compressions performed on the individual 808, and other physiological states of the individual 808. In various examples, the detection circuit 810 includes one or more analog filters configured to filter noise and/or artifact from the resultant signal. The detection circuit 810 generates a digital signal indicative of the impedance using an ADC. This digital signal can be referred to as an “impedance signal” or an “impedance.”

The detection circuit 810 provides the ECG signal and/or the impedance signal one or more processors 812 in the external defibrillator 800. In some implementations, the processor(s) 812 includes a central processing unit (CPU), a graphics processing unit (GPU), both CPU and GPU, or other processing unit or component known in the art.

The processor(s) 812 is operably connected to memory 814. In various implementations, the memory 814 is volatile (such as random-access memory (RAM)), non-volatile (such as read only memory (ROM), flash memory, etc.) or some combination of the two. The memory 814 stores instructions that, when executed by the processor(s) 812, causes the processor(s) 812 to perform various operations. In various examples, the memory 814 stores methods, threads, processes, applications, objects, modules, any other sort of executable instruction, or a combination thereof. In some cases, the memory 814 stores files, databases, or a combination thereof. In some examples, the memory 814 includes, but is not limited to, RAM, ROM, electrically erasable programmable read-only memory (EEPROM), flash memory, or any other memory technology. In some examples, the memory 814 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information, and which can be accessed by the processor(s) 812 and/or the external defibrillator 800. In some cases, the memory 814 at least temporarily stores the ECG signal and/or the impedance signal.

In various examples, the memory 814 includes a detector 816, which causes the processor(s) 812 to determine, based on the ECG signal and/or the impedance signal, whether the individual 808 is exhibiting a particular heart rhythm. For instance, the processor(s) 812 determines whether the individual 808 is experiencing a shockable rhythm that is treatable by defibrillation. Examples of shockable rhythms include ventricular fibrillation (VF) and ventricular tachycardia (V-Tach). In some examples, the processor(s) 812 determines whether any of a variety of different rhythms (e.g., asystole, sinus rhythm, atrial fibrillation (AF), etc.) are present in the ECG signal.

The processor(s) 812 is operably connected to one or more input devices 818 and one or more output devices 820. Collectively, the input device(s) 818 and the output device(s) 820 function as an interface between a user and the defibrillator 800. The input device(s) 818 is configured to receive an input from a user and includes at least one of a keypad, a cursor control, a touch-sensitive display, a voice input device (e.g., a speaker), a haptic feedback device, or any combination thereof. The output device(s) 820 includes at least one of a display, a speaker, a haptic output device, a printer, or any combination thereof. In various examples, the processor(s) 812 causes a display among the input device(s) 818 to visually output a waveform of the ECG signal and/or the impedance signal. In some implementations, the input device(s) 818 includes one or more touch sensors, the output device(s) 820 includes a display screen, and the touch sensor(s) are integrated with the display screen. Thus, in some cases, the external defibrillator 800 includes a touchscreen configured to receive user input signal(s) and visually output physiological parameters, such as the ECG signal and/or the impedance signal.

In some examples, the memory 814 includes an advisor 850, which, when executed by the processor(s) 812, causes the processor(s) 812 to generate advice and/or control the output device(s) 820 to output the advice to a user (e.g., a rescuer). In some examples, the processor(s) 812 provides, or causes the output device(s) 820 to provide, an instruction to perform CPR on the individual 808. In some cases, the processor(s) 812 evaluates, based on the ECG signal, the impedance signal, or other physiological parameters, CPR being performed on the individual 808 and causes the output device(s) 820 to provide feedback about the CPR in the instruction. According to some examples, the processor(s) 812, upon identifying that a shockable rhythm is present in the ECG signal, causes the output device(s) 820 to output an instruction and/or recommendation to administer a defibrillation shock to the individual 808.

The memory 814 also includes an initiator 852 which, when executed by the processor(s) 812, causes the processor(s) 812 to control other elements of the external defibrillator 800 in order to administer a defibrillation shock to the individual 808. In some examples, the processor(s) 812 executing the initiator 852 selectively causes the administration of the defibrillation shock based on determining that the individual 808 is exhibiting the shockable rhythm and/or based on an input from a user (received, e.g., by the input device(s) 818. In some cases, the processor(s) 812 causes the defibrillation shock to be output at a particular time, which is determined by the processor(s) 812 based on the ECG signal and/or the impedance signal.

The memory 814 includes a monitor 848 which, when executed by the processor(s) 812, causes the processor(s) to determine reliability index information and/or determine display settings for data to be displayed on output device(s) 820 and/or external devices 844. The monitor 848 includes the components and/or be configured to perform the functions of the monitoring module 206, monitoring module 222, and/or monitoring module 302 described with respect to FIGS. 2 and 3.

The processor(s) 812 is operably connected to a charging circuit 822 and a discharge circuit 824. In various implementations, the charging circuit 822 includes a power source 826, one or more charging switches 828, and one or more capacitors 830. The power source 826 includes, for instance, a battery. The processor(s) 812 initiates a defibrillation shock by causing the power source 826 to charge at least one capacitor among the capacitor(s) 830. For example, the processor(s) 812 activates at least one of the charging switch(es) 828 in the charging circuit 822 to complete a first circuit connecting the power source 826 and the capacitor to be charged. Then, the processor(s) 812 causes the discharge circuit 824 to discharge energy stored in the charged capacitor across a pair of defibrillation electrodes 834, which are in contact with the individual 808. For example, the processor(s) 812 deactivates the charging switch(es) 828 completing the first circuit between the capacitor(s) 830 and the power source 826, and activates one or more discharge switches 832 completing a second circuit connecting the charged capacitor 830 and at least a portion of the individual 808 disposed between defibrillation electrodes 834.

The energy is discharged from the defibrillation electrodes 834 in the form of a defibrillation shock. For example, the defibrillation electrodes 834 are connected to the skin of the individual 808 and located at positions on different sides of the heart of the individual 808, such that the defibrillation shock is applied across the heart of the individual 808. The defibrillation shock, in various examples, depolarizes a significant number of heart cells in a short amount of time. The defibrillation shock, for example, interrupts the propagation of the shockable rhythm (e.g., VF or V-Tach) through the heart. In some examples, the defibrillation shock is 200 J or greater with a duration of about 0.015 seconds. In some cases, the defibrillation shock has a multiphasic (e.g., biphasic) waveform. The discharge switch(es) 832 are controlled by the processor(s) 812, for example. In various implementations, the defibrillation electrodes 834 are connected to defibrillation leads 836. The defibrillation leads 836 are connected to a defibrillation port 838, in implementations. According to various examples, the defibrillation leads 836 are removable from the defibrillation port 838. For example, the defibrillation leads 836 are plugged into the defibrillation port 838.

In various implementations, the processor(s) 812 is operably connected to one or more transceivers 840 that transmit and/or receive data over one or more communication networks 842. For example, the transceiver(s) 840 includes a network interface card (NIC), a network adapter, a local area network (LAN) adapter, or a physical, virtual, or logical address to connect to the various external devices and/or systems. In various examples, the transceiver(s) 840 includes any sort of wireless transceivers capable of engaging in wireless communication (e.g., radio frequency (RF) communication). For example, the communication network(s) 842 includes one or more wireless networks that include a 3rd Generation Partnership Project (3GPP) network, such as a Long Term Evolution (LTE) radio access network (RAN) (e.g., over one or more LE bands), a New Radio (NR) RAN (e.g., over one or more NR bands), or a combination thereof. In some cases, the transceiver(s) 840 includes other wireless modems, such as a modem for engaging in WI-FI®, WIGIG®, WIMAX®, BLUETOOTH®, or infrared communication over the communication network(s) 842.

The defibrillator 800 is configured to transmit and/or receive data (e.g., ECG data, impedance data, data indicative of one or more detected heart rhythms of the individual 808, data indicative of one or more defibrillation shocks administered to the individual 808, etc.) with one or more external devices 844 via the communication network(s) 842. The external devices 844 include, for instance, mobile devices (e.g., mobile phones, smart watches, etc.), Internet of Things (IoT) devices, medical devices, computers (e.g., laptop devices, servers, etc.), or any other type of computing device configured to communicate over the communication network(s) 842. In some examples, the external device(s) 844 is located remotely from the defibrillator 800, such as at a remote clinical environment (e.g., a hospital). According to various implementations, the processor(s) 812 causes the transceiver(s) 840 to transmit data to the external device(s) 844. In some cases, the transceiver(s) 840 receives data from the external device(s) 844 and the transceiver(s) 840 provide the received data to the processor(s) 812 for further analysis.

In various implementations, the external defibrillator 800 also includes a housing 846 that at least partially encloses other elements of the external defibrillator 800. For example, the housing 846 encloses the detection circuit 810, the processor(s) 812, the memory 814, the charging circuit 822, the transceiver(s) 840, or any combination thereof. In some cases, the input device(s) 818 and output device(s) 820 extend from an interior space at least partially surrounded by the housing 846 through a wall of the housing 846. In various examples, the housing 846 acts as a barrier to moisture, electrical interference, and/or dust, thereby protecting various components in the external defibrillator 800 from damage.

In some implementations, the external defibrillator 800 is an automated external defibrillator (AED) operated by an untrained user (e.g., a bystander, layperson, etc.) and can be operated in an automatic mode. In automatic mode, the processor(s) 812 automatically identifies a rhythm in the ECG signal, decides whether to administer a defibrillation shock, charges the capacitor(s) 830, discharges the capacitor(s) 830, or any combination thereof. In some cases, the processor(s) 812 controls the output device(s) 820 to output (e.g., display) a simplified user interface to the untrained user. For example, the processor(s) 812 refrains from causing the output device(s) 820 to display a waveform of the ECG signal and/or the impedance signal to the untrained user, in order to simplify operation of the external defibrillator 800.

In some examples, the external defibrillator 800 is a monitor-defibrillator utilized by a trained user (e.g., a user, an emergency responder, etc.) and can be operated in a manual mode or the automatic mode. When the external defibrillator 800 operates in manual mode, the processor(s) 812 cause the output device(s) 820 to display a variety of information that is relevant to the trained user, such as waveforms indicating the ECG data and/or impedance data, notifications about detected heart rhythms, and the like.

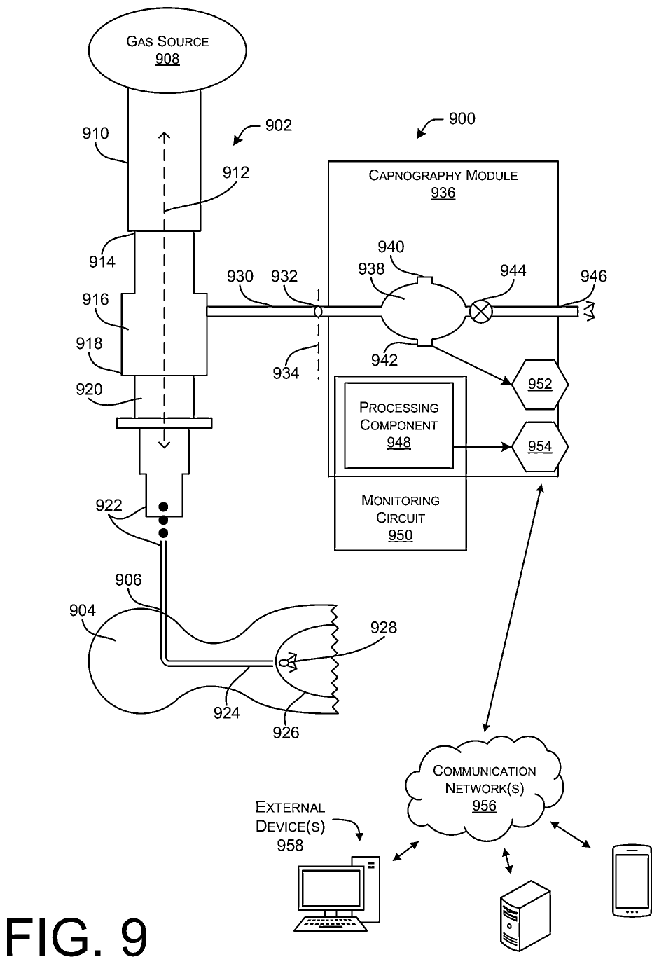

FIG. 9 illustrates an example of a capnography system 900 configured to perform various functions described herein, according to at least one example. FIG. 9 shows a portion of a patient 904, with an airway opening 906 and one of their lungs indicated as 118. A capnography system 900 according to one example is shown, which is configured to be used together with a ventilation system 902 according to embodiments. In some embodiments, the components of the capnography system 900 and system 902 overlap, for better cooperation.

Ventilation system 902 includes a gas source 908. The gas source 908 can be oxygen, air, a mixture thereof, other combinations of gases, etc. Gas source 908 can be configured to expel repeated bursts of the gas through a gas source tube 910. For expelling, gas source 908 is mechanized or manual. In some manual embodiments, gas source 908 includes a bag that can be squeezed by a rescuer, each squeezing delivering one of the bursts.

A ventilation system according to one example includes and/or is configured to work with an airway tube. Examples of airway tubes include an endotracheal (ET) tubes, supraglottic airway laryngopharyngeal tubes, etc. In this description often an ET tube is shown, but that is by way of example and not of limitation, plus aspects of its description for purposes of this document apply also to other types of airway tubes.

Ventilation system 902 also includes an endotracheal (ET) tube 922, of which two portions are shown in different scales, and connected by three dots. In particular, patient 904 is intubated by ET tube 922, and a first portion of ET tube 922 has been inserted through airway opening 906 into the trachea of patient 904. As such, a first end 924 of ET tube 922 is thus brought close to lung 926.

ET tube 922 defines an air path that communicates with gas source tube 910 and gas source 908. As such, when ET tube 922 is thus inserted in the patient's airway, it can be configured so as to guide the bursts 928 of gas as artificial inhalations to lung 926 of patient 904. Gas expulsion by gas source 908 results in a pressure difference between the interior of ET tube 922 and the atmosphere, slightly stretching ET tube 922. The pressure in the interior of ET tube 922 is referred to as the patient's airway pressure and is also closely related to the patient's intrathoracic pressure.

Additional components is coupled between ET tube 922 and gas source 908, in a manner that preserves and accommodates the air path. Such components includes adapters, fittings, valves, etc. Coupling can happen because the components are usually tubular, and circular, and employ a male-female matching configuration.

In the example of FIG. 9 such a component is an airway adapter 916, which is configured to be coupled between gas source 908 and ET tube 922. Airway adapter 916 has a first end 918 that is coupled to a second end of ET tube 922. Airway adapter 916 also has a second end 914 that is coupled to gas source tube 910. Airway adapter 916 has a hollow interior, so as to accommodate the air path when it is thus coupled.

Capnograph system 900 can be configured to detect carbon dioxide in exhalations of patient 904, and also a pressure in the air path. Capnograph system 900 includes a capnography module 936 that has a carbon dioxide detector 942. Carbon dioxide detector 942 can be configured to generate a carbon dioxide signal 952 responsive to an amount of carbon dioxide detected within the air path of ventilation system 902.

Capnograph system 900 also includes a monitoring circuit 950 that is distinct from carbon dioxide detector 942. Monitoring circuit 950 can be configured to detect a pressure in the air path. Monitoring circuit 950 has a processing component 948 within capnography module 936, and distinct from carbon dioxide detector 942. In fact, in some embodiments, monitoring circuit 950 is wholly included within capnography module 936, while in other embodiments not necessarily. Processing component 948 can be configured to generate a pressure signal 954 responsive to the pressure detected in the air path by the monitoring circuit 950. Pressure signal 954, alone or in combination with other signals such as carbon dioxide signal 952, is used to detect spontaneous breaths of the patient.

In some examples, capnography module 936 communicates with the air path by means of a side tube 930, which can be configured to be coupled between airway adapter 916 and capnography module 936. In fact, airway adapter 916 is interposed in the air path for the purpose of providing the opportunity of side tube 930 to access the air path, for sampling the gases and the pressure therein. The gases include a mixture of gases expelled by gas source 908 as bursts, and also from patient 904 as exhalations. For operation, side tube 930 is passed through an opening 932 in a housing 934 of capnography module 936, or of a monitor that houses capnography module 936, a monitor-defibrillator system, etc. In such configurations, capnography module 936 can be characterized as a side stream capnograph.

In some embodiments, capnography module 936 includes a cuvette 938, which is a small chamber. Side tube 930 can be coupled to cuvette 938. Capnography module 936 also includes a pump 944, which is configured to draw gas from the air path into cuvette 938. This way, gases in the air path can be sampled while in cuvette 938. After that, the sampled gases can be disposed of via an exhaust tube 946. It will be understood that pump 944 withdraws, for sampling the vertical column of the air path, relatively little gas compared to what is needed for ventilating the patient. In addition, as long as pump 944 withdraws gas at a constant rate, that will not mask the transient nature of a peak that is intended to be detected.

In such embodiments, a light source 940 in capnography module 936 illuminates the interior of cuvette 938, and carbon dioxide detector 942 detects an amount of the carbon dioxide within cuvette 938, by measuring how much light from light source 940 reaches it. In some embodiments, light source 940 emits infrared (IR) light.

As mentioned above, pressure signal 954, alone or in combination with other signals such as carbon dioxide signal 952, is used by a processor to detect spontaneous breaths of the patient according to embodiments among other physiological parameters, including end-tidal carbon dioxide or other respiratory parameters.

In various implementations, the processing component 948 is operably connected to one or more transceivers that transmit and/or receive the carbon dioxide signal 952, the pressure signal 954, and other signals over one or more communication networks 956. For example, the transceiver(s) includes a network interface card (NIC), a network adapter, a local area network (LAN) adapter, or a physical, virtual, or logical address to connect to the various external devices and/or systems. In various examples, the transceiver(s) includes any sort of wireless transceivers capable of engaging in wireless communication (e.g., radio frequency (RF) communication). For example, the communication network(s) 956 includes one or more wireless networks that include a 3rd Generation Partnership Project (3GPP) network, such as a Long Term Evolution (LTE) radio access network (RAN) (e.g., over one or more LE bands), a New Radio (NR) RAN (e.g., over one or more NR bands), or a combination thereof. In some cases, the transceiver(s) includes other wireless modems, such as a modem for engaging in WI-FI®, WIGIG®, WI MAX®, BLUETOOTH®, or infrared communication over the communication network(s) 956.

The capnography system 900 is configured to transmit and/or receive data (e.g., capnograph data with one or more external devices 958 via the communication network(s) 956. The external devices 958 include, for instance, mobile devices (e.g., mobile phones, smart watches, etc.), Internet of Things (IoT) devices, medical devices, computers (e.g., laptop devices, servers, etc.), or any other type of computing device configured to communicate over the communication network(s) 956. In some examples, the external device(s) 958 is located remotely from the capnography system 900, such as at a remote clinical environment (e.g., a hospital). According to various implementations, the processing component 948 causes the transceiver(s) to transmit data to the external device(s) 958. In some cases, the transceiver(s) receives data from the external device(s) 958 and the transceiver(s) provide the received data to the processing component 948 for further analysis, including analysis of reliability of the data for use as described herein.

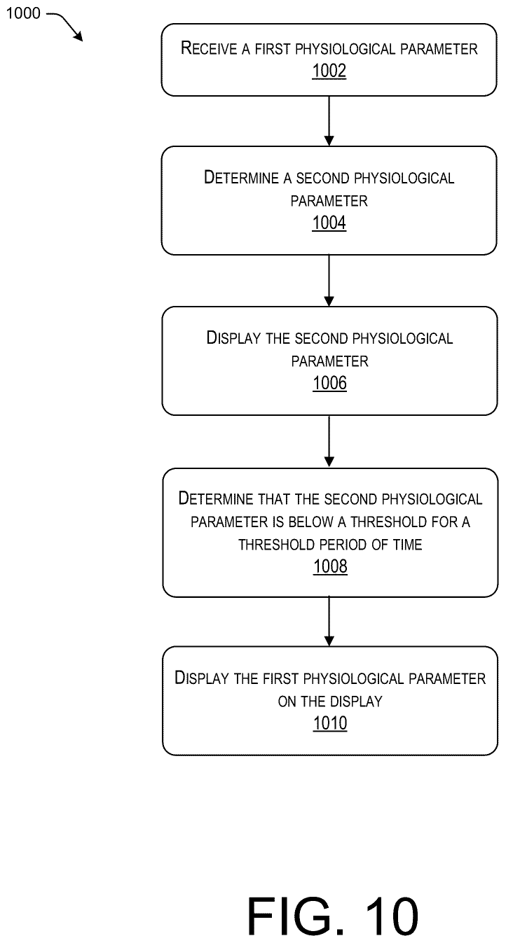

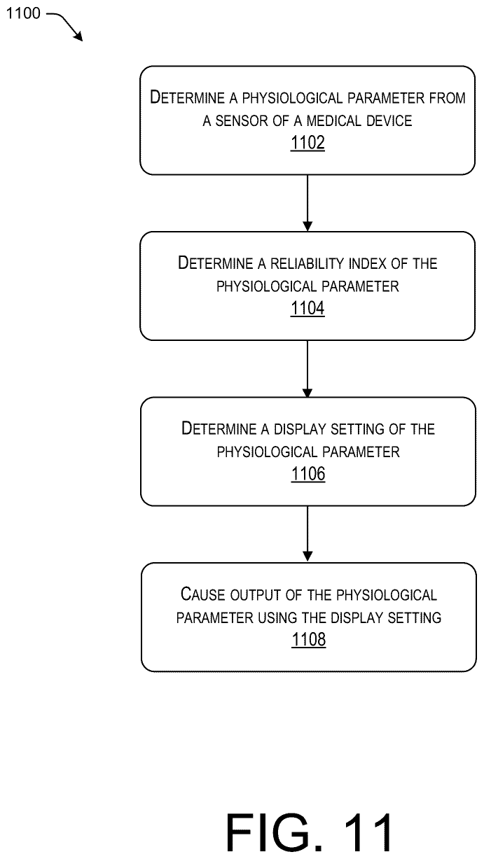

FIGS. 10 and 11 illustrate flow diagrams of methods according to the present technology. For simplicity of explanation, the methods are depicted and described as a series of acts. However, acts in accordance with this disclosure can occur in various orders and/or concurrently, and with other acts not presented and described herein. Furthermore, not all illustrated acts are necessary to implement the methods in accordance with the disclosed subject matter. In addition, those skilled in the art will understand and appreciate that the methods could alternatively be represented as a series of interrelated states via a state diagram or events. Additionally, it should be appreciated that the methods disclosed in this specification are capable of being stored on an article of manufacture to facilitate transporting and transferring such methods to computing devices. The term article of manufacture, as used herein, is intended to encompass a computer program accessible from any computer-readable device or storage media.

Any of a variety of other process implementations which would occur to one of ordinary skill in the art, including but not limited to variations or modifications to the process implementations described herein, are also considered to be within the scope of this disclosure.

FIG. 10 is an example flowchart illustrating a process 1000 for determining to display a first physiological parameter or second physiological parameter on a display associated with a medical device, according to at least one example.

At 1002, the process 1000 includes receiving a first physiological parameter. The first physiological parameter is received from a sensor associated with the medical device and includes data from the sensor as well as derived physiological parameter data. In some examples, the first physiological parameter includes data from multiple sensors associated with the medical device. In an example, the sensor includes a capnography sensor configured to detect a capnograph of the patient. A second sensor detects a respiration rate of the patient. The sensor conveys data associated with the capnography sensor and the respiration rate sensor to a computing device

At 1004, the process 1000 includes determining a second physiological parameter. The second physiological parameter is determined using the first physiological parameter, with the second physiological parameter being a parameter that provides context or understanding of a patient status to a user in a simple and intuitive manner. For instance, at the computing device, the respiration rate and/or capnography data is used to determine end-tidal carbon dioxide for a patient.

At 1006, the process 1000 includes displaying the second physiological parameter. The end-tidal carbon dioxide is displayed on a display. The computing device further causes the display to show the respiration rate data in some examples.

At 1008, the process 1000 includes determining that the second physiological parameter is below a threshold for a threshold period of time. In the example, the respiration rate drops below a threshold level for a threshold period of time, which is a result of sensor error or a result of slowing or stopping respiration of the patient—very different clinical conditions requiring different actions on different timeframes that need to be distinguished to determine if intervention with the patient is warranted. For example, when the respiration rate of the patient detected, drops below five breaths per minute over a threshold period of time of ten to thirty seconds or more. In some examples, other thresholds, such as higher or lower breaths per minute over greater or shorter periods of time is introduced as well. The drop in the respiration rate is associated with a drop in a reliability score for the end-tidal carbon dioxide, or other parameters, as the drop in the respiration rate is a sign of unreliable data.

At 1010, the process 1000 includes displaying the first physiological parameter on the display. In response to the second parameter being below the threshold, the computing device causes the display to output a representation of the capnograph, rather than the derived end-tidal carbon dioxide value. In some examples, the computing device outputs the capnograph on the display in response to reliability data of the second parameter being below a threshold value.

In some examples, the end-tidal carbon dioxide value is removed from the display in response to determining the reliability of the data is below a threshold value for a threshold period of time. For example, the end-tidal carbon dioxide is removed from the display and replaced with a representation of the capnograph data from the sensor. The capnograph data is displayed as a chart, graph, numerical value, or other representation, with an indicator that the display is presenting capnograph data rather than end-tidal carbon dioxide. In some examples, the display of the end-tidal carbon dioxide and the capnograph data is presented on the display in different types of representations (e.g., a numerical value versus a graphical representation) to enable a user to rapidly identify the type of data displayed. In such examples, the capnograph data is presented over a period of time, rather than at an instant point in time, with the capnograph representing data detected at a present time and a previous time while the end-tidal carbon dioxide is presented as an instantaneous numerical value associated with a present value.

In some examples, the display additionally has a feature altered to reflect the change in data presented on the display. For instance, when the capnograph data is presented on the display rather than the end-tidal carbon dioxide, a color of a portion of the display associated with the representation changes, such as to a bright or highly visible color such as red or yellow to draw attention to the change in the displayed data. In some examples, the brightness of the display is also changed, across the entire display, such as to increase a brightness when the capnograph data is presented, and/or by changing a label of data on the display to label the capnograph data as such.

In some examples, the computing device further causes an audio output in response to the determination. The audio signal is altered in response to presenting the capnograph on the display. For instance, the audio signal includes a tone when the capnograph is initially presented. In some examples, the audio signal is a tone that changes in pitch and/or volume in response to displaying the capnograph.

FIG. 11 is an example flowchart illustrating a process 1100 for displaying a reliability indication of physiological parameters, according to at least one example.

At 1102, the process 1100 includes determining a physiological parameter from a sensor of a medical device. the medical device includes a computing device that receives sensor data from a sensor of the medical device and determines a physiological parameter based on the sensor data.

At 1104, the process 1100 includes determining a reliability index of the physiological parameter. The reliability index is a score associated with an accuracy and/or a predicted accuracy of the physiological parameter. In some examples, the computing device determines the reliability index by cross-correlating a first subset of the physiological parameter data with a second subset of the physiological parameter. The cross-correlation is a measure of a similarity of the two subsets and in some examples is performed using a convolution, inner product, correlation matrix, or other such calculations of cross-correlation. In some examples, the computing device includes a machine learning component trained to output the reliability index in response to input of the physiological parameter and/or sensor data from the sensor used to derive the physiological parameter.

At 1106, the process 1100 includes determining a display setting of the physiological parameter. The computing device determines a visibility setting for the physiological parameter on the display and cause the parameter to be displayed using the display setting. The visibility setting reflects a visual clarity of the representation of the physiological parameter on the display. In some examples, the visibility setting for the display includes a visual sharpness and/or blurriness for the representation of the physiological parameter. The visual sharpness scales proportionally with the reliability index such that a lower reliability index is associated with a lower visual sharpness (e.g., a higher blurriness) and a higher reliability index is associated with a higher visual sharpness (e.g., a lower blurriness). The visibility setting also includes a color of the display, a color of a portion of the display, a color of text or numerical representations within the display, a color of a graphical representation, a brightness level of the display, a brightness of a portion of the display, a brightness of text, a brightness of a line of a graphical representation, a contrast level for the display and/or for a text or graphical item, a text size, a pixilation level (e.g. the degree to which text and/or graphical representations appear pixelated, similar to a blurriness), or other such display setting. In some examples, the visibility setting is associated with an audio signal output by the device. The audio signal is scaled similar to the visibility setting, for example to scale a volume, pitch, proportion of static within the audio signal, frequency, tone, or other setting of the audio signal.

At 1108, the process 1100 includes causing output of the physiological parameter using the display setting. The visibility setting and/or display settings used by the external device to display the physiological parameter is used as an indication to a user of the reliability index without presenting an additional numerical value or representation of the reliability index. In this manner, the display is not further cluttered with additional data, but presents additional dimensions of the already presented data in an intuitive manner for users to understand.

### Example Clauses

A. A capnography monitoring device, comprising: a capnography sensor configured to detect a capnograph of a patient; a respiration sensor configured to detect a respiration rate of the patient; a display; a processor electrically coupled to the capnography sensor, the respiration sensor, and the display, the processor being configured to: determine an end-tidal carbon dioxide of the patient by analyzing the capnograph; cause the display to visually output the end-tidal carbon dioxide; cause the display to visually output the respiration rate; determine that the respiration rate is below a threshold level for a threshold period of time; and in response to determining that the respiration rate is below the threshold level for the threshold period of time, cause the display to visually output the capnograph.
B. The capnography monitoring device of clause A, wherein the end-tidal carbon dioxide is displayed at a location on the display, and the processor is configured to display the capnograph by: causing the display to refrain from visually outputting the end-tidal carbon dioxide from at the location; and displaying the capnograph at the location on the display.
C. The capnography monitoring device of clause A or B, wherein the display is configured to visually output the capnograph as a numeric value.
D. The capnography monitoring device of any of clauses A to C, wherein the display is configured to visually output the capnograph as a graphical element, the capnograph detected at a present time and the capnograph detected at a previous time.
E. The capnography monitoring device of any of clauses A to D, wherein the processor is further configured to cause the display to alter a feature in response to causing the display to visually output the capnograph, wherein the feature comprises: a color output by the display; a brightness output by the display; or a portion of a label output by the display.
F. The capnography monitoring device of any of clauses A to E, further comprising: a speaker configured to output an audio signal, wherein the processor is further configured to cause the speaker to alter the audio signal in response to causing the display to visually output the capnograph.
G. The capnography monitoring device of any of clauses A to F, wherein the threshold period of time is in a range of ten to thirty seconds and the threshold level is in a range of zero to five breaths per minute.
H. A medical device, comprising: a sensor configured to detect a first physiological parameter of a patient; a display; and a processor electrically coupled to the sensor and configured to: determine a second physiological parameter of the patient by analyzing the first physiological parameter; cause the display to visually output the second physiological parameter; determine that the second physiological parameter is below a threshold level for at least a threshold period of time; and in response to determining that the second physiological parameter is below the threshold level for at least the threshold period of time, cause the display to visually output the first physiological parameter.
I. The medical device of clause H, wherein the second physiological parameter is determined using a first subset of data representing the first physiological parameter.
J. The medical device of clause H or I, wherein the display is configured to visually output a second subset of the data representing the first physiological parameter, the second subset being different from the first subset.
K. The medical device of any of clauses H to J, wherein the display is configured to visually output the second physiological parameter at a location, and the processor is further configured to cause the display to visually output the first physiological parameter by: causing the display to refrain from visually outputting the second physiological parameter at the location; and causing the display to visually output the first physiological parameter at the location.
L. The medical device of any of clauses H to K, wherein the processor is further configured to cause the display to alter a feature in response to causing the display to visually output the first physiological parameter, wherein the feature comprises: a color output by the display; a brightness output by the display; or a label output on a portion of the display.
M. The medical device of any of clauses H to L, further comprising a speaker configured to output an audio signal, wherein the processor is further configured to cause the speaker to alter the audio signal in response to causing the display to visually output the first physiological parameter.
N. The medical device of any of clauses H to M, further comprising a sensor configured to detect a third physiological parameter of a patient, and wherein the processor is further configured to: determine a second audio signal based on the third physiological parameter and the audio signal; and cause the speaker to output the second audio signal.
O. The medical device of any of clauses H to N, wherein the first physiological parameter comprises: an oxygenation of blood of the patient; a respiration rate of the patient; a capnograph of the patient; a pulse rate of the patient; or an electrocardiogram (ECG) of the patient.
P. The medical device of any of clauses H to O, wherein the display is configured to visually output the second physiological parameter at a first location, and is configured to visually output the first physiological parameter at a second location.
Q. A method, comprising: receiving a first physiological parameter of a patient from a sensor; determining a second physiological parameter of the patient by analyzing the first physiological parameter; outputting the second physiological parameter on a display associated with a medical device; determining that the second physiological parameter is below a threshold level for a threshold period of time; and in response to determining that the second physiological parameter is below the threshold level for the threshold period of time, outputting the first physiological parameter on the display.
R. The method of clause Q, further comprising altering a feature of the display in response to outputting the first physiological parameter on the display, wherein the feature comprises: a color of the display; a brightness of the display; or a label of a portion of the display.
S. The method of clause Q or R, wherein the display is configured to output the second physiological parameter at a location on the display, and wherein outputting the first physiological parameter comprises: causing the display to refrain from outputting the second physiological parameter at the location; and causing the display to output the first physiological parameter at the location.
T. The method of any of clauses Q to S, wherein outputting the first physiological parameter comprises: causing the display to refrain from outputting a numeric value representing the second physiological parameter; and causing the display to output a chart of the first physiological parameter over time.
U. The method of any of clauses Q to T, wherein the first physiological parameter comprises: an oxygenation of blood of the patient; a respiration rate of the patient; a capnograph of the patient; a pulse rate of the patient; or an electrocardiogram (ECG) of the patient.
V. The method of any of clauses Q to U, wherein the second physiological parameter is determined using a first subset of data representing the first physiological parameter.
W. The method of any of clauses Q to V, wherein outputting the first physiological parameter comprises outputting a second subset of the data representing the first physiological parameter, the second subset being different from the first subset.
X. A medical device, comprising: a sensor configured to detect a physiological parameter of a patient; and a processor electrically coupled to the sensor and configured to: determine a reliability index of the physiological parameter, the reliability index being indicative of an accuracy of the physiological parameter; determine a visibility setting for the physiological parameter by analyzing the reliability index, the visibility setting being associated with a visual clarity of a representation of the physiological parameter for visual output on a display; and cause the display to visually output the physiological parameter using the visibility setting.
Y. The medical device of clause X, wherein the processor is configured to determine the reliability index of the physiological parameter by cross-correlating a first segment of data indicative of the physiological parameter with a second segment of the data.
Z. The medical device of clause X or Y, wherein the visibility setting comprises a visual sharpness of the representation of the physiological parameter, and wherein the processor is further configured to: determine that the reliability index has decreased; and in response to determining that the reliability index has decreased, causing the display to decrease the visual sharpness.
AA. The medical device of any of clauses X to Z, wherein the display setting comprises: a blurriness level; a text color; a text brightness level; a text contrast level; a text size; or a text pixilation level.
AB. The medical device of any of clauses X to AA, further comprising a speaker configured to output an audio signal, wherein the processor is further configured to cause the speaker to output the audio signal in response to the reliability index being below a threshold value.
AC. The medical device of any of clauses X to AB, wherein the processor being configured to cause the speaker to output the audio signal comprises determining an audio parameter for the audio signal, wherein the audio parameter is scaled according to the reliability index and comprises: a volume of the audio signal; a pitch of the audio signal; a proportion of static within the audio signal; a frequency of the audio signal; or a tone of the audio signal.
AD. The medical device of any of clauses X to AC, wherein the processor is configured to determine the reliability index by inputting the physiological parameter into a machine learning model configured to output the reliability index and trained using previous physiological parameter data labeled with reliability index metadata.
AE. A medical device, comprising: a sensor configured to detect a physiological parameter; and a processor electrically coupled to the sensor and configured to: determine a reliability index of the physiological parameter by analyzing the physiological parameter; determine a display setting of the physiological parameter by analyzing the reliability index; and cause a display to visually output the physiological parameter using the display setting.
AF. The medical device of clause AE, wherein the processor is configured to determine the reliability index for the physiological parameter by: receiving the physiological parameter over a period of time; dividing the physiological parameter over the period of time into a plurality of segments of data indicative of the physiological parameter; determining a correlation score by cross-correlating a first segment of the data with a second segment of data; and determining the reliability index by analyzing the correlation score.
AG. The medical device of clause AE or AF, wherein the processor is configured to determine the reliability index of the physiological parameter by inputting the physiological parameter into a machine learning model trained to output the reliability index and trained using previous physiological parameters labeled with reliability index metadata.
AH. The medical device of any of clauses AE to AG, the physiological parameter being a first physiological parameter, wherein the processor is configured to determine the reliability index of the first physiological parameter by comparing a second physiological parameter with first physiological parameter, the second physiological parameter being associated with the first physiological parameter.
AI. The medical device of any of clauses AE to AH, wherein the physiological parameter comprises heart rate data of a patient, and wherein the processor is configured to determine the reliability index of the physiological parameter by comparing the heart rate data with heart activity data associated with electrical or mechanical activity of a heart of the patient.
AJ. The medical device of any of clauses AE to AI, wherein the processor is configured to determine the reliability index for the physiological parameter by: determining a noise level of the physiological parameter based on a volatility of the physiological parameter over a period of time; and determining the reliability index by analyzing the volatility of the physiological parameter.
AK. The medical device of any of clauses AE to AJ, the physiological parameter being a first physiological parameter, wherein: the medical device is an external defibrillator; the sensor comprises a detection circuit configured to detect electrical signals indicative of an electrical activity of a heart of an individual; the reliability index is a first reliability index; and wherein the processor is further configured to determine a second physiological parameter by using an artifact removal filter to filter chest compression artifacts from the first physiological parameter; determine a second reliability index for the second physiological parameter; determine a second display setting of the second physiological parameter by analyzing the second reliability index; and cause the display to visually output the second physiological parameter using the second display setting.
AL. The medical device of any of clauses AE to AK, wherein: the processor is configured to determine the display setting by analyzing the second reliability index; and the processor is configured to determine the second display setting by analyzing the first reliability index.
AM. The medical device of any of clauses AE to AL, wherein the display setting or the second display setting comprise: a line thickness for a representation of the physiological parameter or a representation of the second physiological parameter within the display; a data offset distance between the representation of the physiological parameter and the representation of the second physiological parameter within the display; an opacity setting for the representation of the physiological parameter or the representation of the second physiological parameter within the display; a color of the representation of the physiological parameter or the representation of the second physiological parameter within the display; or a contrast between a background color and a first color of the representation of the physiological parameter or a second color of the representation of the second physiological parameter within the display.
AN. A method, comprising: determining a physiological parameter from a sensor of a medical device; determining a reliability index of the physiological parameter by analyzing the physiological parameter; determining a display setting of the physiological parameter by analyzing the reliability index; and causing a display associated with the medical device to visually output physiological parameter using the display setting.
AO. The method of clause AN, wherein determining the reliability index of the physiological parameter comprises: dividing the physiological parameter into a plurality of segments of data indicative of the physiological parameter over time; determining a correlation score by cross-correlating a first segment of the data with a second segment of the data; and determining the reliability index by analyzing the correlation score.
AP. The method of clause AN or AO, wherein determining the reliability index comprises inputting the physiological parameter into a machine learning model trained to output the reliability index and trained using previous physiological parameters labeled with reliability index metadata.
AQ. The method of any of clauses AN to AP, further comprising: determining a second physiological parameter from the sensor; determining a second reliability index for the second physiological parameter by analyzing the second physiological parameter; determining a second display setting for the second physiological parameter by analyzing the second reliability index; and causing the display to visually output the second physiological parameter using the second display setting.
AR. The method of any of clauses AN to AQ, further comprising: determining a global quality score for data associated with a patient by analyzing the reliability index and the second reliability index; and causing the display to visually output the global quality score.
AS. The method of any of clauses AN to AR, wherein the physiological parameter comprises heart rate data of a patient, and wherein determining the reliability index of the physiological parameter comprises comparing the heart rate data with heart activity data associated with electrical or mechanical activity of a heart of the patient.

While the example clauses described above are described with respect to one particular implementation, it should be understood that, in the context of this document, the content of the example clauses can also be implemented via a method, device, system, computer-readable medium, and/or another implementation. Additionally, any of examples A-AS may be implemented alone or in combination with any other one or more of the examples A-AS.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of, or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the implementation to the specified elements, steps, ingredients, or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is warranted, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member is referred to and claimed individually or in any combination with other members of the group or other elements found herein. It is anticipated that one or more members of a group is included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A capnography monitoring device, comprising:
a capnography sensor configured to detect a capnograph of a patient;
a respiration sensor configured to detect a respiration rate of the patient;
a display;
a processor electrically coupled to the capnography sensor, the respiration sensor, and the display, the processor being configured to:
determine an end-tidal carbon dioxide of the patient by analyzing the capnograph;
cause the display to visually output the end-tidal carbon dioxide;
cause the display to visually output the respiration rate;
determine that the respiration rate is below a threshold level for a threshold period of time, the threshold level being in a range of zero to five breaths per minute, the threshold period of time being in a range of ten to thirty seconds; and
in response to determining that the respiration rate is below the threshold level for the threshold period of time, cause the display to visually output the capnograph.

2. The capnography monitoring device of claim 1, wherein the processor is further configured to cause the display to alter a feature in response to causing the display to visually output the capnograph, wherein the feature comprises:
a color output by the display;
a brightness output by the display; or
a portion of a label output by the display.

3. The capnography monitoring device of claim 1, further comprising:
a speaker configured to output an audio signal,
wherein the processor is further configured to cause the speaker to alter the audio signal in response to causing the display to visually output the capnograph.

4. A medical device, comprising:
a sensor configured to detect a first physiological parameter of a patient;
a display; and
a processor electrically coupled to the sensor and configured to:
determine a second physiological parameter of the patient by analyzing the first physiological parameter;
cause the display to visually output the second physiological parameter;
determine that the second physiological parameter is below a threshold level for at least a threshold period of time; and
in response to determining that the second physiological parameter is below the threshold level for at least the threshold period of time, cause the display to visually output the first physiological parameter.

5. The medical device of claim 4, wherein the second physiological parameter is determined using a first subset of data representing the first physiological parameter.

6. The medical device of claim 5, wherein the display is configured to visually output a second subset of the data representing the first physiological parameter, the second subset being different from the first subset.

7. The medical device of claim 4, wherein the display is configured to visually output the second physiological parameter at a location, and the processor is further configured to cause the display to visually output the first physiological parameter by:
causing the display to refrain from visually outputting the second physiological parameter at the location; and
causing the display to visually output the first physiological parameter at the location.

8. The medical device of claim 4, wherein the processor is further configured to cause the display to alter a feature in response to causing the display to visually output the first physiological parameter, wherein the feature comprises:
a color output by the display;
a brightness output by the display; or
a label output on a portion of the display.

9. The medical device of claim 4, further comprising a speaker configured to output an audio signal, wherein the processor is further configured to cause the speaker to alter the audio signal in response to causing the display to visually output the first physiological parameter.

10. The medical device of claim 9, further comprising a sensor configured to detect a third physiological parameter of a patient, and wherein the processor is further configured to:
determine a second audio signal based on the third physiological parameter and the audio signal; and
cause the speaker to output the second audio signal.

11. The medical device of claim 4, wherein the first physiological parameter comprises:
an oxygenation of blood of the patient;
a respiration rate of the patient;
a capnograph of the patient;
a pulse rate of the patient; or
an electrocardiogram (ECG) of the patient.

12. The medical device of claim 4, wherein the display is configured to visually output the second physiological parameter at a first location, and is configured to visually output the first physiological parameter at a second location.

13. A method, comprising:
receiving a first physiological parameter of a patient from a sensor;
determining a second physiological parameter of the patient by analyzing the first physiological parameter;
outputting the second physiological parameter on a display associated with a medical device;
determining that the second physiological parameter is below a threshold level for a threshold period of time; and
in response to determining that the second physiological parameter is below the threshold level for the threshold period of time, outputting the first physiological parameter on the display.

14. The method of claim 13, further comprising altering a feature of the display in response to outputting the first physiological parameter on the display, wherein the feature comprises:
a color of the display;
a brightness of the display; or
a label of a portion of the display.

15. The method of claim 13, wherein the display is configured to output the second physiological parameter at a location on the display, and wherein outputting the first physiological parameter comprises:
causing the display to refrain from outputting the second physiological parameter at the location; and
causing the display to output the first physiological parameter at the location.

16. The method of claim 13, wherein outputting the first physiological parameter comprises:
causing the display to refrain from outputting a numeric value representing the second physiological parameter; and
causing the display to output a chart of the first physiological parameter over time.

17. The method of claim 13, wherein the first physiological parameter comprises:
an oxygenation of blood of the patient;
a respiration rate of the patient;
a capnograph of the patient;
a pulse rate of the patient; or
an electrocardiogram (ECG) of the patient.

18. The method of claim 13, wherein the second physiological parameter is determined using a first subset of data representing the first physiological parameter.

19. The method of claim 18, wherein outputting the first physiological parameter comprises outputting a second subset of the data representing the first physiological parameter, the second subset being different from the first subset.

20. The method of claim 13, wherein the second physiological parameter comprises a heart rate, a respiration rate, or an end-tidal carbon dioxide of the patient.
